(19) United States
(12) Patent Application Publication
(10) Pub. No.: US 2014/0322717 A1
(43) Pub. Date: Oct. 30, 2014

(54) SELECTIVE AMPLIFICATION AND DETECTION OF MUTANT GENE ALLELES

(71) Applicant: THE TRUSTEES OF COLUMBIA UNIVERSITY IN THE CITY OF NEW YORK, New York, NY (US)

(72) Inventors: Dingbang Xu, New York, NY (US); Charles Emala, Woodcliff Lake, NJ (US)

(21) Appl. No.: 14/358,004

(22) PCT Filed: Nov. 16, 2012
(86) PCT No.: PCT/US2012/065606

§ 371 Date: May 13, 2014

Related U.S. Application Data
(60) Provisional application No. 61/561,703, filed on Nov. 18, 2011.
(60) Provisional application No. 61/673,933, filed on Jul. 20, 2012.

(51) Int. Cl. C12Q 1/68

(52) U.S. Cl. CPC C12Q 1/6806
USPC 435/6.12; 435/91.2; 536/24.33

(57) ABSTRACT

The present invention provides methods for selectively amplifying a mutant allele of a gene. These methods include (a) contacting a sample containing a mutant and a normal allele of a gene with at least one primer that selectively modifies the normal allele of the gene and causes further amplification of the normal allele to fail or to be substantially reduced; and (b) selectively amplifying the mutant allele of the gene or a portion thereof. Other methods for detecting a mutant allele of a gene in a sample are also provided.

### CROSS-REFERENCE TO RELATED APPLICATIONS

This application claims benefit to U.S. Provisional Patent Application Ser. Nos. 61/561,703 filed Nov. 18, 2011 and 61/673,933, filed Jul. 20, 2012. The contents of the above application are incorporated by reference as if recited in full.

### FIELD OF INVENTION

The present invention provides, inter alia, methods for selectively amplifying a mutant allele and enhancing the sensitivity of detection of gene mutations.

### INCORPORATION BY REFERENCE OF SEQUENCE LISTING

This application contains references to amino acids and/or nucleic acid sequences that have been filed concurrently herewith as sequence listing text file “0344404.txt”, file size of 20 KB, created on Nov. 15, 2012. The aforementioned sequence listing is hereby incorporated by reference in its entirety pursuant to 37 C.F.R. §1.52(e)(5).

### BACKGROUND OF THE INVENTION

A large number of solid tumor cancers, including breast cancer, are caused by single point mutations or small base pair insertions/deletions in susceptible genes. The detection of these mutations typically relies on the polymerase chain reaction (PCR) followed by a variety of post-amplification analyses. However, a major limitation of all of these methods is their low sensitivities since amplification of fewer copies of the abnormal gene is competitively inhibited by preferential amplification of the more abundant normal gene. In fact, detection of the abnormal gene is often not possible until it represents greater than 5-10% of the total alleles present meaning that early detection of a low number of cancer cells is severely limited.

Accordingly, there is a need for selective amplification of and early detection of mutant gene alleles present in low abundance in, inter alia, breast and other solid organ cancers. The present invention is directed to meeting this and other needs.

### SUMMARY OF THE INVENTION

Disclosed herein are methods that modify standard nucleic acid amplification methods such as, e.g., PCR reactions to specifically and significantly block the amplification of a normal gene and thus enhance the detection of limited copies of a mutant allele. One of the methods involves the selective degradation of a normal allele using thermostable restriction endonucleases before and/or during the PCR reaction. Another method involves the design of primers that, when extended, create amplicons of the normal allele that cannot be used for further amplification as well as mutant amplicons that can be further amplified.

Accordingly, one embodiment of the present invention is a method for selectively amplifying a mutant allele. This method comprises:

(a) contacting a sample comprising a mutant and a normal allele of a gene with at least one primer that selectively modifies the normal allele of the gene and causes further amplification of the normal allele to fail or to be substantially reduced; and

(b) selectively amplifying the mutant allele of the gene or a portion thereof.

Another embodiment of the present invention is a method for detecting a mutant allele of a gene, if present, in a sample. This method comprises:

(a) contacting the sample with a thermostable restriction endonuclease that recognizes a sequence in a normal allele of the gene but not in a mutant allele of the gene;

(b) amplifying the mutant allele of the gene or a portion thereof, if present in the sample; and

(c) before or during step (b), selectively degrading the normal allele of the gene.

Yet another embodiment of the present invention is a method for detecting a mutant allele of a gene, if present, in a sample using a PCR. This method comprises:

(a) contacting the sample with a first primer that produces a first and a second amplicon after two rounds of amplification, the first amplicon being an amplicon of the normal allele and cannot serve as a template for subsequent amplification, and the second amplicon being an amplicon of the mutant allele; and

(b) contacting the sample with a second primer that anneals to the second amplicon.

A further embodiment of the present invention is a template tool for use in selecting optimal PCR conditions for a method of detecting a mutant allele of a gene in a sample, which method selectively degrades a normal allele of the gene in the sample by a thermostable restriction endonuclease. This template tool comprises:
a first polynucleotide sequence comprising recognition sites for a plurality of thermostable endonucleases, and
a second polynucleotide sequence homologous to the first polynucleotide except that it does not contain a single recognition site for any one of the plurality of thermostable endonucleases.

Another embodiment of the present invention is a template tool for use in selecting optimal PCR conditions for a method of selectively amplifying mutant alleles present in low abundance in solid tumors, which method selectively degrades a normal allele or an amplicon of the normal allele in the sample by a thermostable restriction endonuclease. This template tool comprises:

a first polynucleotide sequence comprising recognition sites for a plurality of thermostable endonucleases, and
a second polynucleotide sequence homologous to the first polynucleotide except that it does not contain a single recognition site for any one of the plurality of thermostable endonucleases.

An additional embodiment of the present invention is a kit. This kit comprises any template tool disclosed herein.

### BRIEF DESCRIPTION OF THE DRAWINGS

FIG. 1 is a schematic showing that the normal allele and its amplicons are degraded before and during lower-denaturation-temperature PCR by a thermostable restriction endonuclease that recognizes a sequence natively present or introduced by primer design.

FIG. 2 shows a DNA gel demonstrating that a 466 basepair (bp) fragment from mouse adenylate cyclase subtype 5 and a 178 bp fragment from mouse glyceraldehyde 3-phosphate dehydrogenase (GAPDH) can be effectively amplified under denaturing temperatures of 90° C. and 87.5° C., respectively. Annealing and extension were performed at 72° C. for both reactions.

FIG. 3 shows a DNA gel demonstrating that a 185 bp fragment from human β-actin can be effectively amplified under a denaturing temperature of 85° C. Annealing and extension were performed at 55° C. and 70° C., respectively.

FIG. 4 shows a DNA gel demonstrating that a 179 bp fragment from human β-actin can be effectively amplified under a denaturing temperature of 81° C. Annealing and extension were performed at 55° C. and 70° C., respectively.

FIG. 5 shows a DNA gel demonstrating that a 131 bp fragment from human chromosome 6 (AL731777/GI29801752) can be effectively amplified under denaturing temperatures of 72.5° C.-75.0° C. Annealing and extension were both performed at 60° C. The forward primer used for this reaction was CATTTACATCTTTCAATTATGTATATTCTT (SEQ. ID NO: 49), and the reverse primer was ATGCAAATACTTCATTAGTAAAATTTACTT (SEQ. ID NO: 50)

FIG. 6 shows a DNA gel demonstrating that PhoI, but not MwoI or BstNI, can retain activity under a 92° C. denaturing step and effectively block the amplification of a 388 bp fragment from human GAPDH. The fragment contained PhoI (GGCC), MwoI (GCNNNNNNNGC), and BstNI (CCWGG) restriction sites. The amplification was performed using brain cDNA with an Advantage 2 PCR kit (Clontech Laboratories Inc., Mountain View, Calif.). Denaturing, annealing, and extension temperatures were 92° C., 60° C., and 75° C., respectively.

FIG. 7 shows a DNA gel demonstrating that MwoI can retain activity under a 83° C.-88° C. denaturing step and effectively block the amplification of a 86 bp fragment from human guanylate cyclase 2F containing a MwoI (GCNNNNNNNGC) restriction site. The amplification was performed using brain cDNA with an Advantage 2 PCR kit. Annealing and extension temperatures were 60° C. and 70° C., respectively.

FIG. 8 shows a DNA gel demonstrating that a 131 bp fragment from human chromosome 6 can be effectively amplified under a denaturing temperature of 66.4° C. with 20% glycerol in the reaction mixture. The same fragment can be effectively amplified alternatively under a denaturing temperature of 69.0° C. with 10% glycerol in the reaction mixture. Annealing and extension temperatures were 35° C. and 60° C., respectively.

FIG. 9 is a schematic showing the extension of a primer carrying a sequence that enhances hairpin formation during the PCR amplification of a normal allele to allow for the selective amplification of a mutant allele.

FIG. 10 is a PCR product analysis of rat (as mutant) and mouse (as wild) cDNA mixed at the given ratio. The PCR product was digested with either XbaI or Tru9I.

FIG. 11 is a table showing primer design and thermostable restriction endonuclease selection for enhanced detection of specific breast cancer mutations.

FIG. 12 is a table showing primer design to induce amplicon hairpin formation from normal alleles to allow enhanced detection of breast cancer mutations.

FIG. 13 is a bar graph showing the distribution of PCR primer lengths (cited from Yuryev, Anton. PCR Primer Design, Humana Press, 2007.).

FIG. 14 is a bar graph showing the distribution of PCR product sizes for all primer pairs in the VirOligo database (Oklahoma State University, Stillwater, Okla.) (Yuryev, Anton. PCR Primer Design, Humana Press, 2007.).

FIG. 15 is a bar graph showing the distribution of melting temperatures (Tₘ) for approximately 100 randomly selected amplification products from ten mRNA sequences (NM_{—}000901-NM_{—}000910). Products were selected with automatic searching using Oligo software, version 7 (Molecular Biology Insights, Inc., Cascade, Colo.).

FIG. 16 is a scatter plot and regression line showing the relationship between Tₘ and PCR product length using data from FIG. 15.

FIG. 17 is a line graph showing that Tₘ is lower in PCR products with low guanine-cytosine (G-C) content.

FIG. 18 is a line graph showing the relationship between Tₘ and PCR product length.

FIG. 19 shows the amplification plots of synthetic B-raf templates representing wild type or mutant sequences of the B-raf gene in the absence or presence of the thermostable enzyme TspRI.

FIG. 20 shows the DNA sequencing results of PCR products following amplification of a wild type and a mutant template of the B-raf gene present in a 1,000:1 ratio.

FIGS. 21A-21G show the amplification plots of wild type genomic DNA in the absence or presence of thermostable restriction endonucleases known to digest wild type regions of several genes that commonly encode mutations in solid tumors.

FIG. 22 shows the DNA sequencing results following enriched PCR amplification of a synthetic mutant site in a set of custom synthesized universal templates, which allows for the identification of optimal conditions for the activities of thermostable enzymes. Examples of two experiments are presented. A mixture of a wild type universal template and a mutant universal template were mixed in a 100:1 ratio and subjected to PCR amplification at a reduced denaturation temperature (85° C.) in the presence of BseLI (top tracing) and Bse JI (bottom tracing). These enzymes successfully degraded the wild type amplification products allowing for the equal detection of the mutant product (top tracing) or the detection of only the mutant product (bottom tracing).

### DETAILED DESCRIPTION OF THE INVENTION

One embodiment of the present invention is a method for selectively amplifying a mutant allele. This method comprises:

(a) contacting a sample comprising a mutant and a normal allele of a gene with at least one primer that selectively modifies the normal allele of the gene and causes further amplification of the normal allele to fail or to be substantially reduced; and

(b) selectively amplifying the mutant allele of the gene or a portion thereof.

As used herein, a “normal allele” is a variation of a gene that is the most prevalent in a population or a subpopulation of interest, such as e.g., the wild type. A “mutant allele” is a variation of the gene other than the normal allele.

In this invention, a sample, which comprises nucleic acids, may be obtained from an organism, such as a mammal, for example, a human, a farm animal, a laboratory animal, or a pet. Some examples of farm animals include cows, pigs, horses, goats, etc. Some examples of pets include dogs, cats, etc. Some examples of laboratory animals include rats, mice, rabbits, guinea pigs, etc.

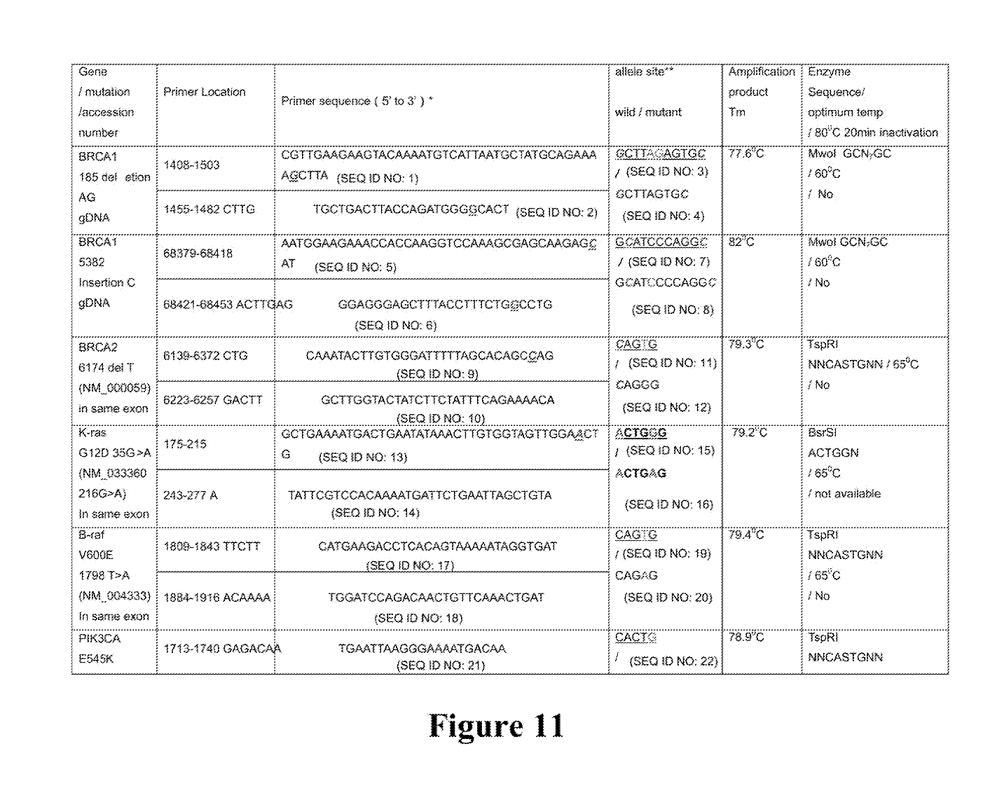

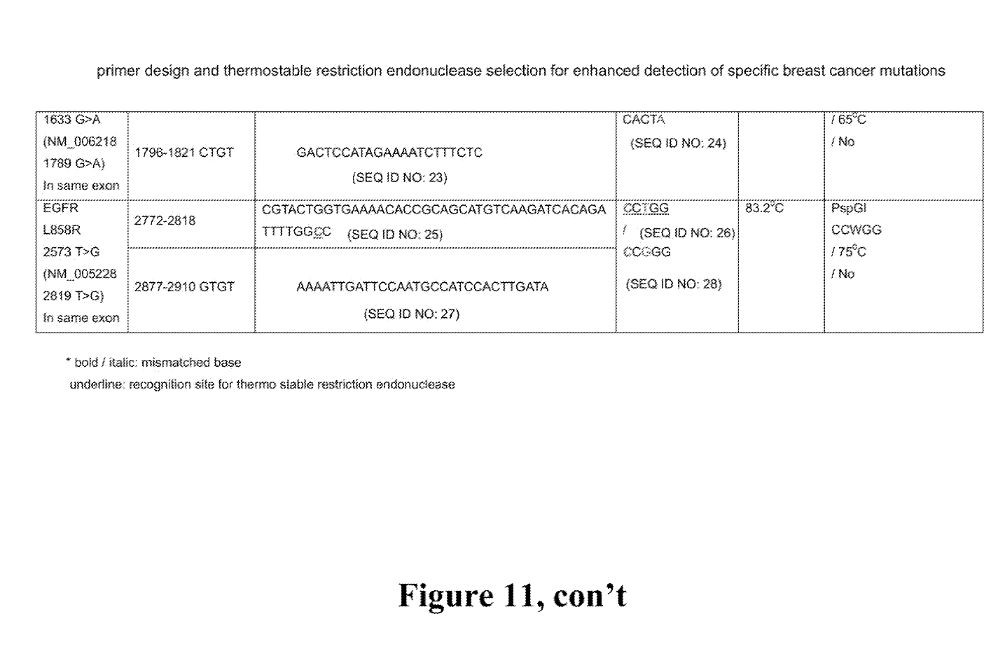

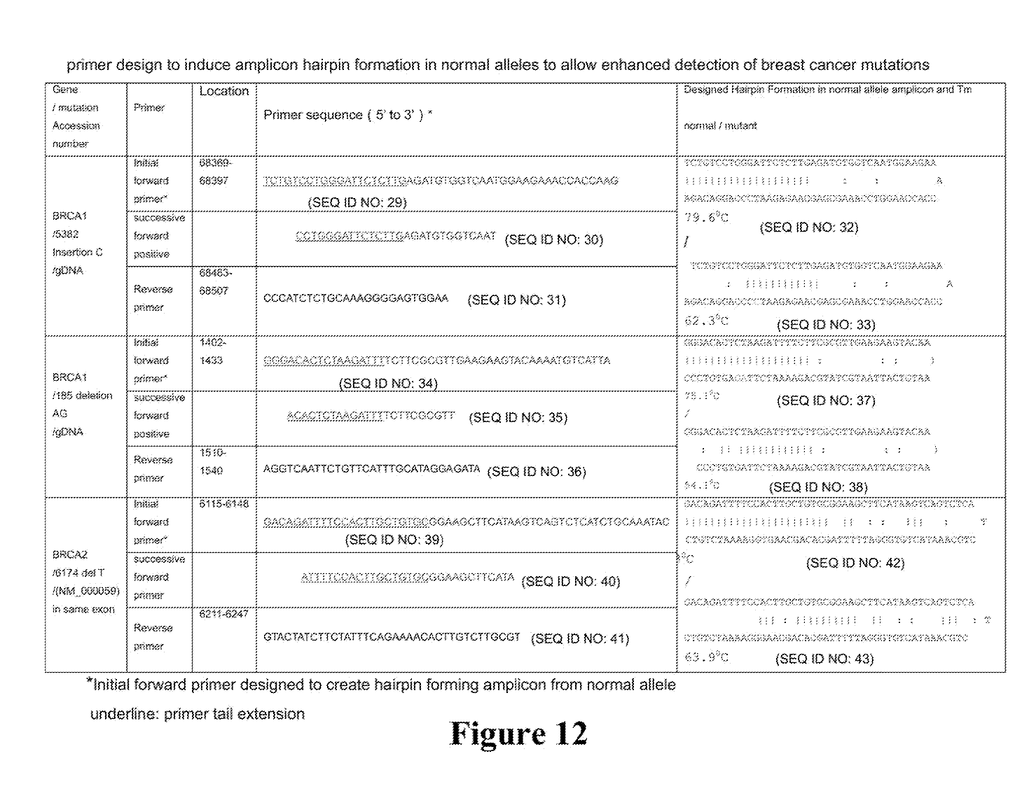

As used herein, a “primer” is a short strand of nucleic acid, which (or a portion of which) anneals to a gene of interest or a portion thereof. The primer serves as a starting point for the amplification of the gene or a portion thereof. Exemplary sequences of primers suitable for use in the methods of the present invention include those disclosed in FIGS. 11 and 12 (such as, e.g., SEQ ID NOs: 1-2, 5-6, 9-10, 13-14, 17-18, 21, 23, 25, 27, 29-31, 34-36, and 39-41). Primers include forward and reverse primers. A forward primer initiates elongation of a nucleic acid strand from the 5′ end to the 3′ end. Reverse primers initiate the elongation of a nucleic acid strand from the 3′ end to the 5′ end.

In the present method, amplification step(s) may be carried out using any suitable technique known in the art. Non-limiting examples of methods for amplification of nucleic acids include PCR, ligation amplification (or ligase chain reaction (LCR)), amplification methods based on the use of Q-beta replicase or template-dependent polymerase (see, e.g., US Patent Publication Number US20050287592); helicase-dependent isothermal amplification (Vincent et al., “Helicase-dependent isothermal DNA amplification”. EMBO reports 5 (8): 795-800 (2004)); strand displacement amplification (SDA); thermophilic SDA nucleic acid sequence based amplification (3SR or NASBA) and transcription-associated amplification (TAA). Preferably, the amplification step(s) of the present invention are carried out with PCR.

In the present invention, selective modification of the normal allele of a gene by a primer includes, for example, introducing a thermostable restriction endonuclease site into the normal allele after the first two rounds of PCR, or causing an amplicon of the normal allele to form a hairpin, for example, a hairpin in the 3′ end of the amplicon, after the first two rounds of PCR. As used herein, an “amplicon” means a nucleic acid product formed as a result of an amplification process, such as, e.g., PCR. As used herein, each “round” of PCR is a cycle of denaturation of double-stranded nucleic acids, annealing of the primers to the single-stranded nucleic acids, and extension of the primers. As used herein, a “thermostable” restriction endonuclease means a restriction enzyme that retains its activity or a portion thereof after exposure to elevated temperature (e.g., greater than about 70° C.). Preferably, the thermostable restriction endonuclease retains its activity or a portion thereof after exposure to temperatures of greater than about 80° C. Non-limiting examples of restriction endonucleases for use in accordance with the methods of the present invention include Ack1, Apa LI, Ape KI, Bam HI, Bam HI-HF, Bcl I, Bgl II, Blp I, Bsa AI, Bsa XI, BseJI, BseLI, BseSI, Bsi HKAI, BsmI, Bso BI, BsrI, Bsr FI, BsrSI, Bst BI, Bst EII, BstHHI, BstSF1, Bst NI, Bst UI, Bst Z17I, Bts CI, CspCI, Cvi QI, EsaBC3I, EsaBC4I, Hpa I, Hyp188I, Kpn I, MjaI, MjaII, MjaIII, MjaIV, MjaV, MspNI, Mwo I, Nci I, PabI, Pae R7I, Pho I, Ppu MI, PspGI, Pvu II, Sfi I, Sfo I, SmlI, SuiI, TaaI, TaiI, TaqI, Tti I, Taq52, TasI, TatI, TauI, TceI, TfiI, TfiL, TliI, TmaI, TneI, Tru1I, TscAI, TseI, Tsp1I, Tsp32I, Tsp321I, Tsp4C, Tsp8E, Tsp49I, TspMI, Tsp45C, Tsp45I, Tsp504I, Tsp505I, Tsp507I, Tsp509I, Tsp510I, Tsp514I, Tsp560I, TspAI, TspDTI, TspGWI, Tsp MI, TspRI, TteI, Tth111I, Tth111II, Tth24I, TthHB27I, TthHB8I, TthRQI, TtmI, TtmII, TtrI and Zra I. Combinations of any of the foregoing endonucleases are also contemplated herein.

In one aspect of this embodiment, amplification is carried out with a PCR, which reaction includes denaturing, extension, and annealing steps.

Preferably, the primer introduces a thermostable restriction endonuclease site into an amplicon of the normal allele but not an amplicon of the mutant allele after two rounds of PCR, and selectively amplifying the mutant allele of the gene or a portion thereof is accomplished by contacting the sample with a thermostable restriction endonuclease that recognizes the site introduced into the amplicon of the normal allele.

In this embodiment, the thermostable restriction endonuclease may be active in a PCR reaction mix. As used herein, “active”, with respect to restriction endonucleases, means having the ability to digest DNA at the recognition site. A PCR reaction mix is well known in the art. PCR reaction mixes can be purchased from numerous vendors or mixed in the laboratory. Typically, a PCR reaction mix comprises several components, including, a DNA polymerase, deoxynucleotides, a buffer solution, the PCR template, forward and reverse primers, and water.

The denaturing step of the PCR may be carried out at a temperature that does not substantially irreversibly inactivate the thermostable restriction endonuclease. For example, the denaturing step of the PCR may be carried out at a temperature between about 70° C. and about 90° C., such as about 70° C., 71° C., 72° C., 73° C., 74° C., 75° C., 76° C., 77° C., 78° C., 79° C., 80° C., 81° C., 82° C., 83° C., 84° C., 85° C., 86° C., 87° C., 88° C., 89° C., and 90° C. Ranges within any of these points are also contemplated.

Methods of lowering the temperature of the denaturing step in a PCR reaction are known in the art, such as, e.g., those disclosed below in the Examples section. For instance, co-solvents such as, e.g., glycerol, DMSO, NMP, formamide, and combinations thereof, may be used to lower the temperature of the denaturing step. Furthermore, shortening the length of the PCR product, shortening the length of the primers, and/or lowering the guanine-cytosine (G-C) content of the PCR product may also be used effectively to lower the denaturing temperature.

The annealing step and/or the extension step of the PCR may be carried out at a temperature at which the thermostable restriction endonuclease is active.

For example, the annealing step of the PCR may be carried out at a temperature between about 55° C. and about 72° C., such as about 55° C., 56° C., 57° C., 58° C., 59° C., 60° C., 61° C., 62° C., 63° C., 64° C., 65° C., 66° C., 67° C., 68° C., 69° C., 70° C., 71° C., and 72° C. Ranges within any of these points are also contemplated.

Additionally, the extension step of the PCR may be carried out at a temperature between 60° C. and 72° C., such as 60° C., 61° C., 62° C., 63° C., 64° C., 65° C., 66° C., 67° C., 68° C., 69° C., 70° C., 71° C., and 72° C. Ranges within any of these points are also contemplated.

In a further aspect of this embodiment, the thermostable restriction endonuclease is selected from the group consisting of ApeKI, BclI, BgLII, BlpI, BsaXI, BseJI, BseLI, BseSI, BsiHkAI, BsmI, BsoBI, BsR FI, BsrI, BstBI, BstEII, BstHHI, BstNI, BstSF1, BstUI, BstZ17I, BtsCI, CspCI, EsaBC3I, EsaBC4I, Hyp188I, MjaI, MjaII, MjaIII, MjaIV, MjaV, MspNI, MwoI, PabI, PhoI, PspGI, SfiI, SmlI, SmlI, SuiI, TaaI, TaiI, TaqI, TasI, TatI, TauI, TceI, TfiI, TfiL, TliI, TmaI, TneI, Tru1I, TscAI, TseI, Tsp1I, Tsp32I, Tsp32II, Tsp45C, Tsp45I, Tsp504I, Tsp505I, Tsp507I, Tsp509I, Tsp510I, Tsp514I, Tsp560I, TspAI, TspDTI, TspGWI, TspRI, TteI, Tth111I, Tth111II, Tth24I, TthHB27I, TthHB8I, TthRQI, TtmI, TtmII, and TtrI. Preferably, the thermostable restriction endonuclease is selected from ApeKI, BsaXI, BseJI, BseLI, BsmI, BsrI, BstBI, BstHHI, Hyp188I, MwoI, PhoI, PspGI, TaqI, TasI, TfiI, TseI, Tsp45C, Tsp45I, Tsp509I, and TspRI.

In another aspect of this embodiment, the amount of the mutant allele is at least about 0.02% of the normal allele in the sample such as, e.g., at least about 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 1.5%, 2%, 2.5%, 3%, 4%, 5%, 6%, 7%, 8%, 9%, or 10% of the normal allele in the sample. Ranges within any of these points are also contemplated.

In yet another aspect of this embodiment, the mutant allele is a marker for a cancer or other disease state. Non-limiting examples of cancers for which the mutant allele is a marker include, e.g., adrenocortical carcinoma, anal cancer, bladder cancer, bone cancer, brain tumor, breast cancer, carcinoid tumor, carcinoma, cervical cancer, colon cancer, endometrial cancer, esophageal cancer, extrahepatic bile duct cancer, Ewing family of tumors, extracranial germ cell tumor, eye cancer, gallbladder cancer, gastric cancer, germ cell tumor, gestational trophoblastic tumor, head and neck cancer, hypopharyngeal cancer, islet cell carcinoma, kidney cancer, laryngeal cancer, leukemia, lip and oral cavity cancer, liver cancer, lung cancer, lymphoma, malignant mesothelioma, Merkel cell carcinoma, mycosis fungoides, myelodysplastic syndrome, myeloproliferative disorders, nasopharyngeal cancer, neuroblastoma, oral cancer, oropharyngeal cancer, osteosarcoma, ovarian epithelial cancer, ovarian germ cell tumor, pancreatic cancer, paranasal sinus and nasal cavity cancer, parathyroid cancer, penile cancer, pituitary cancer, plasma cell neoplasm, prostate cancer, rhabdomyosarcoma, rectal cancer, renal cell cancer, transitional cell cancer of the renal pelvis and ureter, salivary gland cancer, Sezary syndrome, skin cancer (such as cutaneous t-cell lymphoma, Kaposi's sarcoma, and melanoma), small intestine cancer, soft tissue sarcoma, stomach cancer, testicular cancer, thymoma, thyroid cancer, urethral cancer, uterine cancer, vaginal cancer, vulvar cancer, Wilms' tumor. Preferably, the cancer is a breast cancer or other solid tumor cancer, such as lung cancer and colon cancer.

In yet another aspect of this embodiment, the mutant allele is selected from the group consisting of 185 deletion AG in BRCA1, 5382 insertion C in BRCA1, 6174 deletion T (6174 del T) in BRCA2, G12D 35G>A/T/C in K-ras, 38G>A/T/C in K-ras, V600E 1798 T>A in B-raf, E545K 1633 G>A in PIK3CA, 1624G>A in PIK3CA, 3140A>G in PIK3C1, and L858R 2573 T>G in EGFR.

In yet another aspect of this embodiment, a first forward primer produces a first and a second amplicon after two rounds of amplification, the first amplicon being an amplicon of the normal allele, which cannot serve as a template for subsequent amplification, and the second amplicon being an amplicon of the mutant allele.

In one preferred embodiment, the sample is contacted with a second forward primer that anneals to the second amplicon. Preferably, the ratio of the first forward primer to the second forward primer is less than 1:10, such as 1:20, 1:30, 1:40, 1:50, 1:60, 1:70, 1:80, 1:90, 1:100, 1:200, 1:300, 1:400, 1:500, 1:600, 1:700, 1:800, 1:900, 1:1000, 1:1500, 1:2000.

In another aspect of the present embodiment, the first amplicon forms a hairpin.

In another aspect of the present embodiment, the first forward primer is selected from the group consisting of TCTGTCCTGGGATTCTCTTGAGATGTGGTCAATGGAAGAAACCACCAAG (SEQ ID NO: 29), GGGACACTCTAAGATTTTCTTCGCGTTGAAGAAGTACAAAATGTCATTA (SEQ ID NO: 34), and GACAGATTTTCCACTTGCTGTGCGGAAGCTTCATAAGTCAGTCTCATCTGCAAA TAC (SEQ ID NO: 39); and the second forward primer is selected from the group consisting of CCTGGGATTCTCTTGAGATGTGGTCAAT (SEQ ID NO: 30), ACACTCTAAGATTTTCTTCGCGTT (SEQ ID NO: 35), and ATTTTCCACTTGCTGTGCGGAAGCTTCATA (SEQ ID NO: 40), respectively.

In a further aspect of the present embodiment, the mutant allele is selected from the group consisting of 5382 insertion C in BRCA1, 185 deletion AG in BRCA1, and 6174 del T in BRCA2.

Another embodiment of the present invention is a method for detecting a mutant allele of a gene, if present, in a sample. The method comprises:

(a) contacting the sample with a thermostable restriction endonuclease that recognizes a sequence in a normal allele of the gene but not in a mutant allele of the gene;

(b) amplifying the mutant allele of the gene or a portion thereof, if present in the sample; and

(c) before or during step (b), selectively degrading the normal allele of the gene.

In this embodiment, the mutant allele is present in the sample, as a percentage of the normal allele, as set forth above. Preferably, the mutant allele is present in the sample in an amount that is at least about 0.02% of the normal allele. In addition, suitable thermostable restriction endonucleases for use in this embodiment are as disclosed above.

In one aspect of this embodiment, the amplification step comprises using a PCR, which reaction includes denaturing, extension, and annealing steps. Preferably, the denaturing step of the PCR is carried out at a temperature that does not substantially irreversibly inactivate the thermostable restriction endonuclease. Furthermore, the annealing step and/or the extension step of the PCR is preferably carried out at a temperature at which the thermostable restriction endonuclease is active. Suitable temperatures for each step are as disclosed above.

In another aspect of this embodiment, the sequence in the normal allele recognized by the thermostable restriction endonuclease is natively present in the normal allele. Alternatively, the sequence recognized by the restriction endonuclease may be introduced by a primer used to amplify a gene or a portion thereof. In the latter case, the sequence recognized by the restriction endonuclease will be introduced after the first two rounds of amplification. Non-limiting examples of the primers used in these reactions are disclosed in FIG. 11.

In yet another aspect of this embodiment, the mutant allele of the gene is a marker for a cancer or other disease state. Various types of cancer that may be detected using the methods of the invention are as disclosed previously.

In yet another aspect of this embodiment, the mutant allele is selected from the group consisting of 185 deletion AG in BRCA1, 5382 insertion C in BRCA1, 6174 del T in BRCA2, G12D 35G>A/T/C in K-ras, 38G>A/T/C in K-ras, V600E 1798 T>A in B-raf, E545K 1633 G>A in PIK3CA, 1624G>A in PIK3CA, 3140A>G in PIK3C1, and L858R 2573 T>G in EGFR.

As set forth above, the source of the sample may be obtained from any organism, such as a mammal, e.g., a human, a farm animal, a laboratory animal, or a pet.

Yet another embodiment of the present invention is a method for detecting a mutant allele of a gene, if present, in a sample using a PCR. The method comprises:

(a) contacting the sample with a first primer that produces a first and a second amplicon after two rounds of amplification, the first amplicon being an amplicon of the normal allele and cannot serve as a template for subsequent amplification, and the second amplicon being an amplicon of the mutant allele; and

(b) contacting the sample with a second primer that anneals to the second amplicon.

In one aspect of this embodiment, the first and the second primers are forward primers. The ratios of the first forward primer to the second forward primer are as disclosed above. Preferably, the ratio of the first forward primer to the second forward primer is less than 1:10.

In another aspect of this embodiment, the first amplicon forms a hairpin.

In an additional aspect of this embodiment, the first primer is selected from the group consisting of TCTGTCCTGGGATTCTCTTGAGATGTGGTCAATGGAAGAAACCACCAAG (SEQ ID NO: 29), GGGACACTCTAAGATTTTCTTCGCGTTGAAGAAGTACAAAATGTCATTA (SEQ ID NO: 34), and GACAGATTTTCCACTTGCTGTGCGGAAGCTTCATAAGTCAGTCTCATCTGCAAA TAC (SEQ ID NO: 39); and the second primer is selected from the group consisting of CCTGGGATTCTCTTGAGATGTGGTCAAT (SEQ ID NO: 30), ACACTCTAAGATTTTCTTCGCGTT (SEQ ID NO: 35), and ATTTTCCACTTGCTGTGCGGAAGCTTCATA (SEQ ID NO: 40), respectively.

In a further aspect of this embodiment, the mutant allele is selected from the group consisting of 5382 insertion C in BRCA1, 185 deletion AG in BRCA1, and 6174 del T in BRCA2.

Another embodiment of the present invention is a template tool for use in selecting optimal PCR conditions for a method of detecting a mutant allele of a gene in a sample, which method selectively degrades a normal allele of the gene in the sample by a thermostable restriction endonuclease. This template tool comprises:
a first polynucleotide sequence comprising recognition sites for a plurality of thermostable endonucleases, and
a second polynucleotide sequence homologous to the first polynucleotide except that it does not contain a single recognition site for any one of the plurality of thermostable endonucleases.

As used herein, PCR conditions include the temperature and the duration of the various steps of the PCR, such as the denaturing step, the annealing step, and the extension step, as well as buffer selection for the PCR.

As used herein, “homologous” means two or more sequences that have at least 50%, 60% identity, preferably 70%, 75%, 80%, 85%, 90%, 91%, 92%, 93%, 94%, 95%, 96%, 97%, 98%, 99%, or higher identity when compared and aligned for maximum correspondence. Methods of aligning sequences for comparison are known in the art. (See, e.g., Smith & Waterman, Adv. Appl. Math. 2:482 (1981); Needleman & Wunsch, J. Mol. Biol. 48:443 (1970), Pearson & Lipman, Proc. Nat'l. Acad. Sci. USA 85:2444 (1988)). In this aspect of the present invention, in the second polynucleotide sequence, such recognition site may contain 1 or more mutations, or differences, in nucleotide sequence, such that each recognition site is non-functional or substantially non-functional. The first and second polynucleotides of this aspect of the invention are exemplified by the so-called “wildtype” and mutant sequences or templates, respectively, disclosed in Example 3 herein.

In one aspect of this embodiment, the first polynucleotide sequence comprises recognition sites for 10-50 different thermostable endonucleases, such as 15-45 different thermostable endonucleases, 20-40 different thermostable endonucleases, 25-35 different thermostable endonucleases, including 26, 27, 28, 29, 30, 31, 32, 33, 34, or 35 different thermostable endonucleases. Preferably, the first polynucleotide sequence comprises recognition sites for 30 different thermostable endonucleases.

In another aspect of this embodiment, the plurality of thermostable endonucleases are selected from the group consisting of any thermostable endonucleases disclosed herein or that are subsequently selected according to at least the following criteria: (1) the enzyme can survive in environments that are greater than 80° C. for at least 20 minutes; and (2) the organism from which the enzyme originates grows in environments having a temperature between 50-70° C. Preferably, the plurality of thermostable endonucleases are selected from the group consisting: TasI, Tru1I, TspDTI, BstNI, PspGI, TscAI, TspRI, PhoI, TaaI, BseLI, MwoI, TseI, ApeKI, BtsCI, TfiI, Tsp45I, BstSF1, SmlI, TatI, TaiI, BsiHkAI, BseSI, BstHHI, TaqI, TspGWI, TauI, BclI, BstUI, Tth111I, BseJI, and combinations thereof.

In a further aspect of this embodiment, the recognition site for each of the plurality of thermostable endonucleases is selected from the group consisting of: AATT, TTAA, ATGAA, CCWGG, CCWGG, CASTG, CASTG, GGCC, ACNGT, CCNNNNNNNGG, GCNNNNNNNGC, GCWGC, GCWGC, GGATG, GAWTC, GTSAC, CTRYAG, CTYRAG, WGTACW, ACGT, GWGCWC, GKGCMC, GCGC, TCGA, ACGGA, GCSGC, TGATCA, CGCG, GACNNNGTC, GATNNNNATC, and combinations thereof.

In an additional aspect of this embodiment, the first and the second polynucleotide sequences are from about 100 base pairs to about 600 base pairs in length, such as about 150 to about 550 base pairs, about 200 to about 500 base pairs, about 250 to about 450 base pairs, or about 300 to about 400 base pairs in length. Preferably, the first and the second polynucleotide sequences are about 300 base pairs in length. Also preferably, the first and the second polynucleotide sequences are the same length.

In another aspect of this embodiment, the melting temperature of the first and the second polynucleotide sequences are from about 75° C. to about 90° C., including from about 76° C. to about 88° C., from about 78° C. to about 85° C., or from about 80° C. to about 83° C. Preferably, the melting temperature of the first and the second polynucleotide sequences are about 80° C. Also preferably, the melting temperature of the first and the second polynucleotide sequences are the same. As used herein, the “melting temperature” of a polynucleotide means the temperature at which 50% of the oligonucleotide and its perfect complement are in duplex. Methods of determining melting temperature are known in the art. The most accurate method is to carry out the determination empirically, but various studies have derived accurate equations for melting temperature using thermodynamic basis sets for nearest neighbor interactions. (See, e.g., Breslauer, K. J.; Frank, R.; Bl\check{s}cker, H.; Marky, L. A. Proc. Natl. Acad. Sci. USA 83, 3746-3750(1986)).

In an additional aspect of this embodiment, the first polynucleotide sequence comprises a sequence as depicted in SEQ ID NO:69 and the second polynucleotide sequence comprises a sequence as depicted in SEQ ID NO:70.

Another embodiment of the present invention is a kit. This kit comprises any template tool disclosed herein. The kit may optionally include directions for its use, buffers and other reagents for facilitating use of the template tool. Furthermore, each of the first and second polynucleotide sequences may be packaged together or separately. Each of the polynucleotide sequences may be packaged in any convenient form, e.g., they may be lyophilized or in solution.

This template tool may be used in conjunction with other methods disclosed herein, including for example, a method for detecting a mutant allele of a gene, if present, in a sample. The method comprises:

(a) contacting the sample with a thermostable restriction endonuclease that recognizes a sequence in a normal allele of the gene but not in a mutant allele of the gene;

(b) amplifying the mutant allele of the gene or a portion thereof, if present in the sample; and

(c) before or during step (b), selectively degrading the normal allele of the gene.

As set forth above, the amplification step (step (b)) may comprise using a PCR, which reaction includes denaturing extension, and annealing steps. Thus, conditions for these PCR steps may be selected by using a template tool according to the present invention, which comprises:
a first polynucleotide sequence comprising recognition sites for a plurality of thermostable endonucleases, and
a second polynucleotide sequence homologous to the first polynucleotide except that it does not contain a single recognition site for any one of the plurality of thermostable endonucleases.

Another embodiment of the present invention is a template tool for use in selecting optimal PCR conditions for a method of selectively amplifying mutant alleles present in low abundance in solid tumors, which method selectively degrades a normal allele or an amplicon of the normal allele in the sample by a thermostable restriction endonuclease, the template tool comprising:
a first polynucleotide sequence comprising recognition sites for a plurality of thermostable endonucleases, and
a second polynucleotide sequence homologous to the first polynucleotide except that it does not contain a single recognition site for any one of the plurality of thermostable endonucleases.

As used herein, “low abundance” means that the mutant allele is less than 50% of the normal allele, including less than 20%, less than 10%, less than 5%, less than 2%, less than 1%, less than 0.5%, less than 0.1%, less than 0.05%, and less than 0.02%.

In one aspect of this embodiment, the first polynucleotide sequence comprises recognition sites for any number of different thermostable endonucleases, including, e.g., for 10-50 different thermostable endonucleases, such as 15-45 different thermostable endonucleases, 20-40 different thermostable endonucleases, 25-35 different thermostable endonucleases, including 26, 27, 28, 29, 30, 31, 32, 33, 34, or 35 different thermostable endonucleases. Preferably, the first polynucleotide sequence comprises recognition sites for 30 different thermostable endonucleases

In another aspect of this embodiment, the plurality of thermostable endonucleases are selected from the group consisting of any thermostable endonucleases disclosed herein or as subsequently selected using as least the criteria set forth above. Preferably, the plurality of thermostable endonucleases are selected from the group consisting: TasI, Tru1I, TspDTI, BstNI, PspGI, TscAI, TspRI, PhoI, TaaI, BseLI, MwoI, TseI, ApeKI, BtsCI, TfiI, Tsp45I, BstSF1, SmlI, TatI, TaiI, BsiHkAI, BseSI, BstHHI, TaqI, TspGWI, TauI, BclI, BstUI, Tth111I, BseJI, and combinations thereof.

In a further aspect of this embodiment, the recognition site for each of the plurality of thermostable endonucleases is selected from the group consisting of: AATT, TTAA, ATGAA, CCWGG, CCWGG, CASTG, CASTG, GGCC, ACNGT, CCNNNNNNNGG, GCNNNNNNNGC, GCWGC, GCWGC, GGATG, GAWTC, GTSAC, CTRYAG, CTYRAG, WGTACW, ACGT, GWGCWC, GKGCMC, GCGC, TCGA, ACGGA, GCSGC, TGATCA, CGCG, GACNNNGTC, GATNNNNATC, and combinations thereof.

In an additional aspect of this embodiment, the first and the second polynucleotide sequences are from about 100 base pairs to about 600 base pairs in length, such as about 150 to about 550 base pairs, about 200 to about 500 base pairs, about 250 to about 450 base pairs, or about 300 to about 400 base pairs in length. Preferably, the first and the second polynucleotide sequences are about 300 base pairs in length. Also preferably, the first and the second polynucleotide sequences are the same length.

In another aspect of this embodiment, the melting temperature of the first and the second polynucleotide sequences are from about 75° C. to about 90° C., including from about 76° C. to about 88° C., from about 78° C. to about 85° C., or from about 80° C. to about 83° C. Preferably, the melting temperature of the first and the second polynucleotide sequences are about 80° C. Also preferably, the melting temperature of the first and the second polynucleotide sequences are the same.

In an additional aspect of this embodiment, the first polynucleotide sequence comprises a sequence as depicted in SEQ ID NO:69 and the second polynucleotide sequence comprises a sequence as depicted in SEQ ID NO:70.

As used herein, the terms “nucleic acid,” “oligonucleotide,” and “polynucleotide” are used interchangeably. In the present invention, these terms mean at least two nucleotides covalently linked together, or any artificially constructed molecules that mimic such a chain. Nucleic acids include without limitation, deoxyribonucleic acid (DNA), ribonucleic acid (RNA), peptide nucleic acid (PNA), morpholino and locked nucleic acid (LNA), glycol nucleic acid (GNA) and threose nucleic acid (TNA).

Nucleic acids may be single stranded or double stranded, or may contain portions of both double stranded and single stranded sequences. As noted above, the nucleic acid may be DNA, both genomic and cDNA, RNA, or a hybrid, where the nucleic acid may contain combinations of deoxyribo- and ribo-nucleotides, and combinations of bases including uracil, adenine, thymine, cytosine, guanine, inosine, xanthine, hypoxanthine, isocytosine and isoguanine. Nucleic acids may be synthesized as a single stranded molecule or expressed in a cell (in vitro or in vivo) using a synthetic gene. Nucleic acids may be obtained by chemical synthesis methods or by recombinant methods.

As noted above, the nucleic acid may also be a RNA such as a mRNA, tRNA, shRNA, siRNA, Piwi-interacting RNA, pri-miRNA, pre-miRNA, miRNA, or anti-miRNA, as described, e.g., in U.S. patent application Ser. Nos. 11/429,720, 11/384,049, 11/418,870, and Ser. No. 11/429,720 and Published International Application Nos. WO 2005/116250 and WO 2006/126040.

A nucleic acid will generally contain phosphodiester bonds, although nucleic acid analogs for use, e.g., in the primers of the present invention, may be included that may have at least one different linkage, e.g., phosphoramidate, phosphorothioate, phosphorodithioate, or O-methylphosphoroamidite linkages and peptide nucleic acid backbones and linkages. Other analog nucleic acids include those with positive backbones; non-ionic backbones, and non-ribose backbones, including those disclosed in U.S. Pat. Nos. 5,235,033 and 5,034,506. Nucleic acids containing one or more non-naturally occurring or modified nucleotides are also included within the definition of nucleic acid.

In this invention, any suitable method for detecting amplified nucleic acids may be used. For example, the amplified nucleic acid species may be sequenced directly using any suitable nucleic acid sequencing method, such as conventional dideoxy sequencing methods, pyrosequencing, nanopore based sequencing methods (e.g., sequencing by synthesis), sequencing by ligation, sequencing by hybridization, and microsequencing (primer extension based polymorphism detection).

The following examples are provided to further illustrate the methods of the present invention. These examples are illustrative only and are not intended to limit the scope of the invention in any way.

### EXAMPLES

### Example 1

### Selective Amplification of Low-Quantity Mutant Alleles by Inclusion of Restriction Endonucleases in PCR Reaction

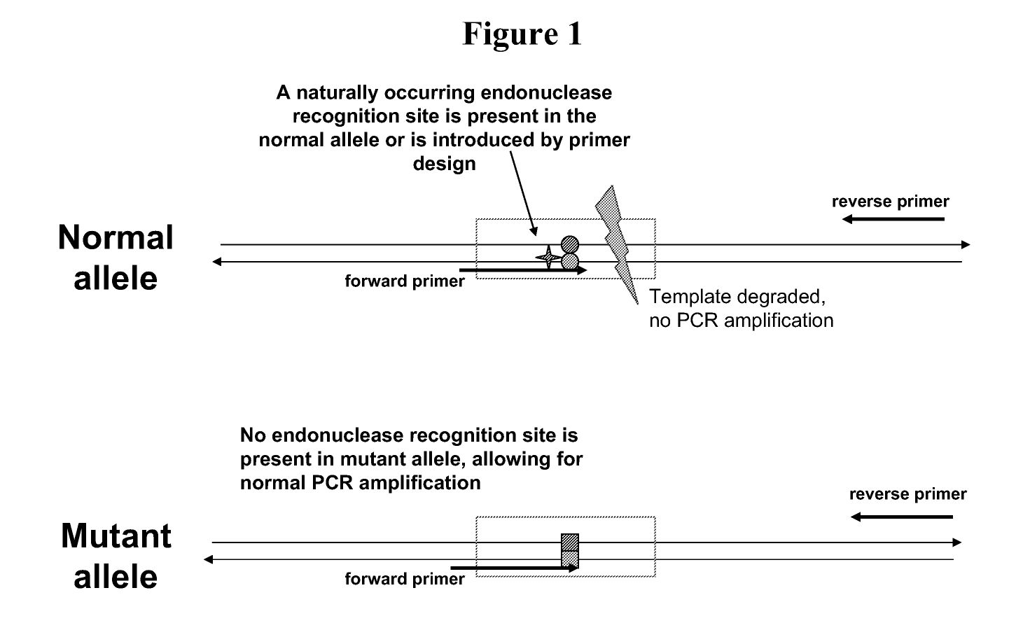

As illustrated in FIG. 1, to selectively amplify the mutant gene allele, one strategy includes (1) primer design that allows amplified products to denature at lower temperatures and creates a thermostable restriction endonuclease site in normal alleles lacking endogenous restriction sites; (2) inclusion of a thermostable endonuclease chosen to destroy the amplicon of the normal gene during each PCR cycle; (3) a change in the typical PCR denaturation step (95° C./30 seconds) to an optimized lower temperature and shorter duration that does not destroy endonuclease activity but does allow denaturation of amplified dsDNA products; and (4) inclusion of co-solvents that reduce dsDNA denaturation temperatures from the typical 94° C. or 95° C. and enhance enzyme thermo-stability.

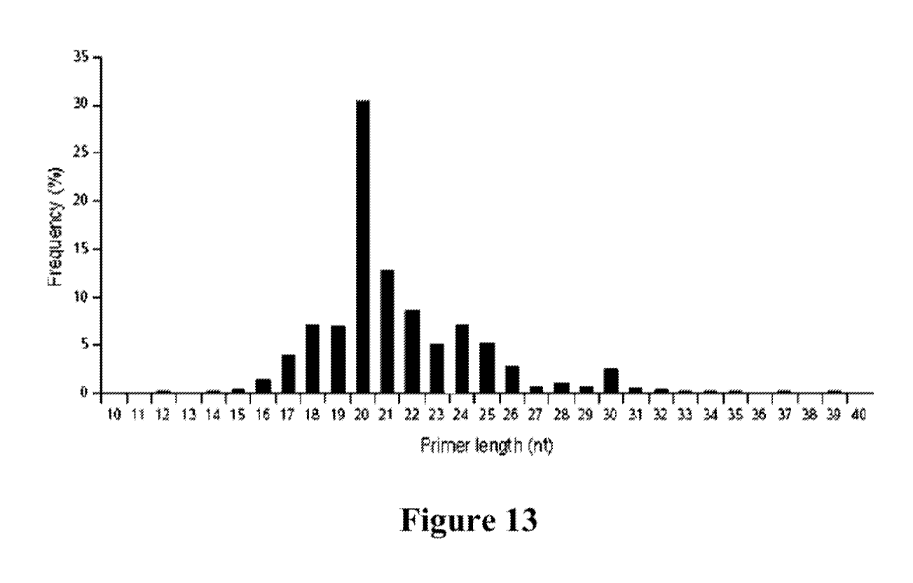

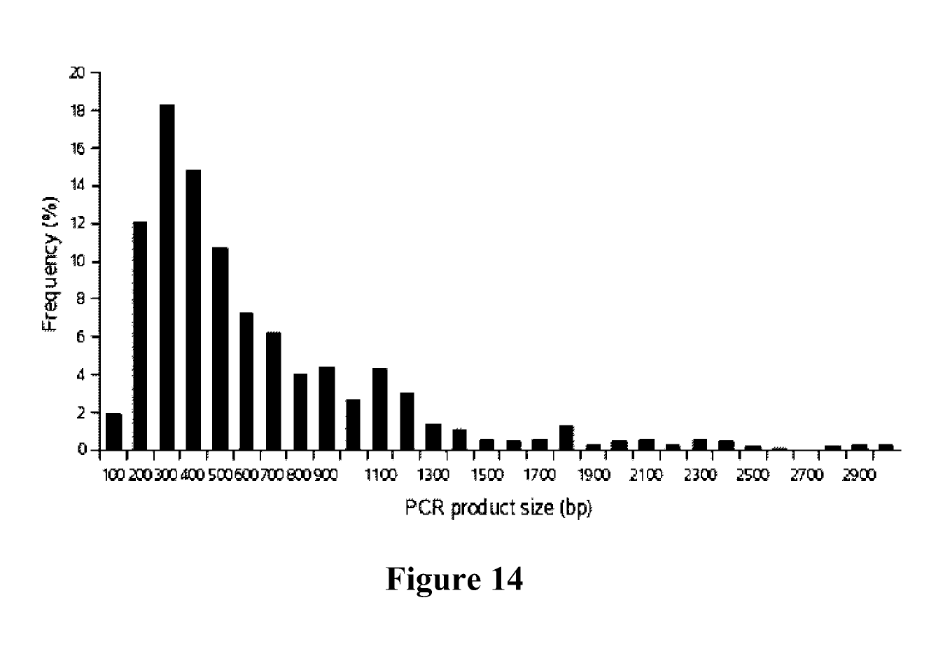

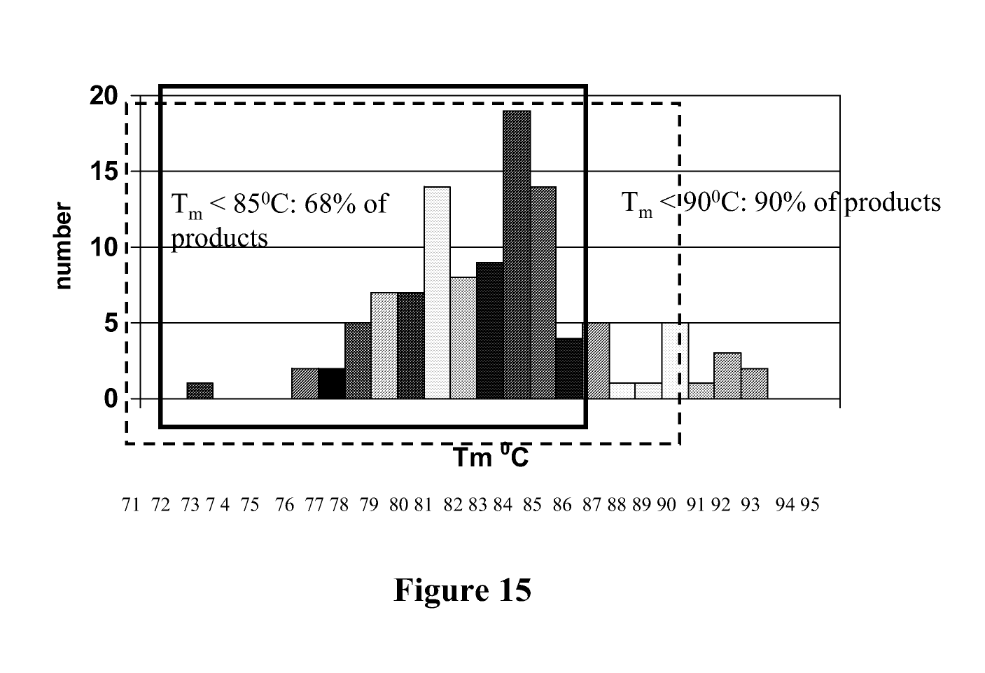

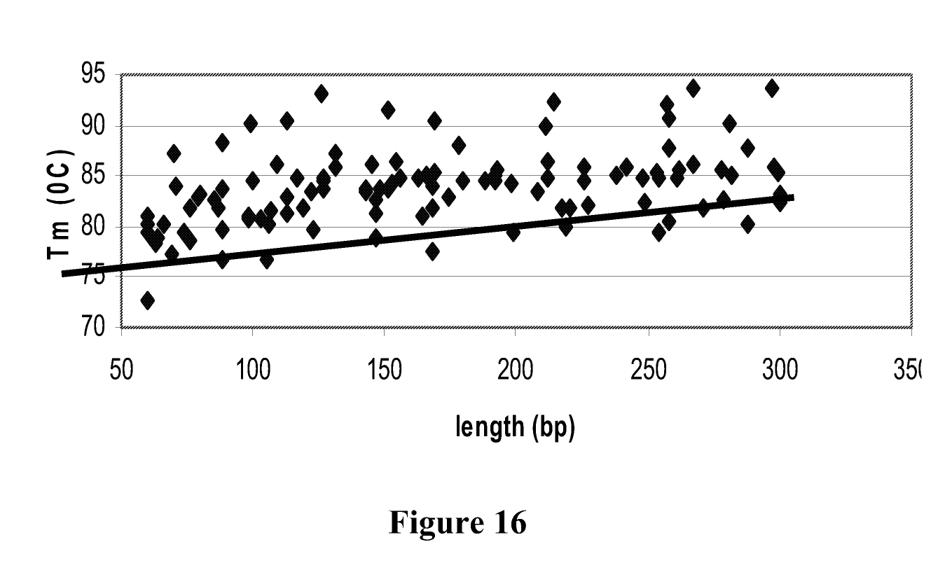

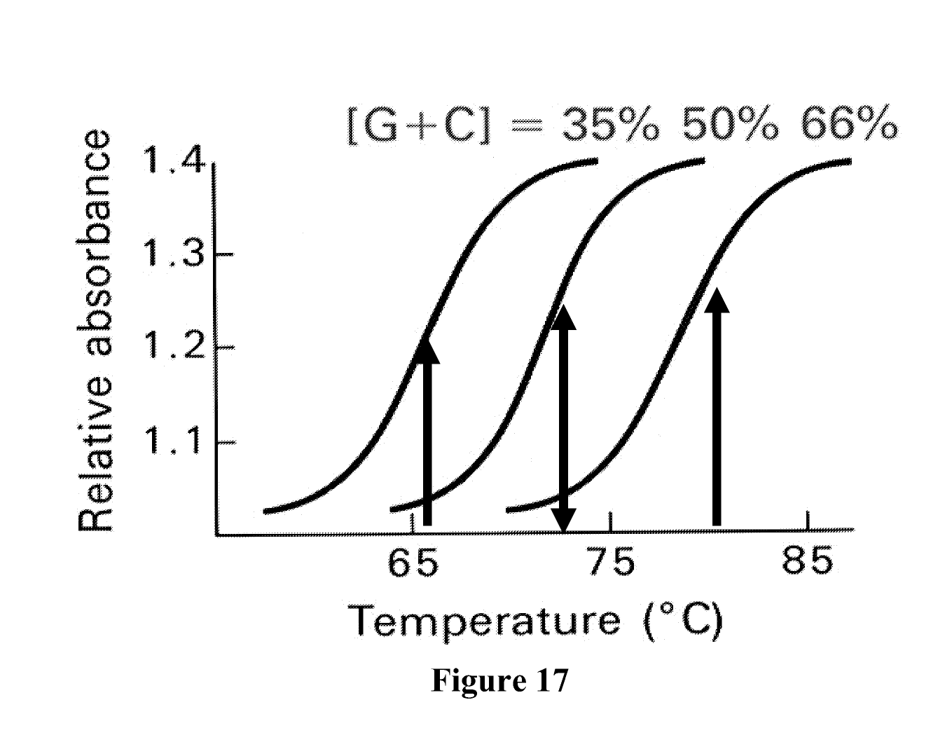

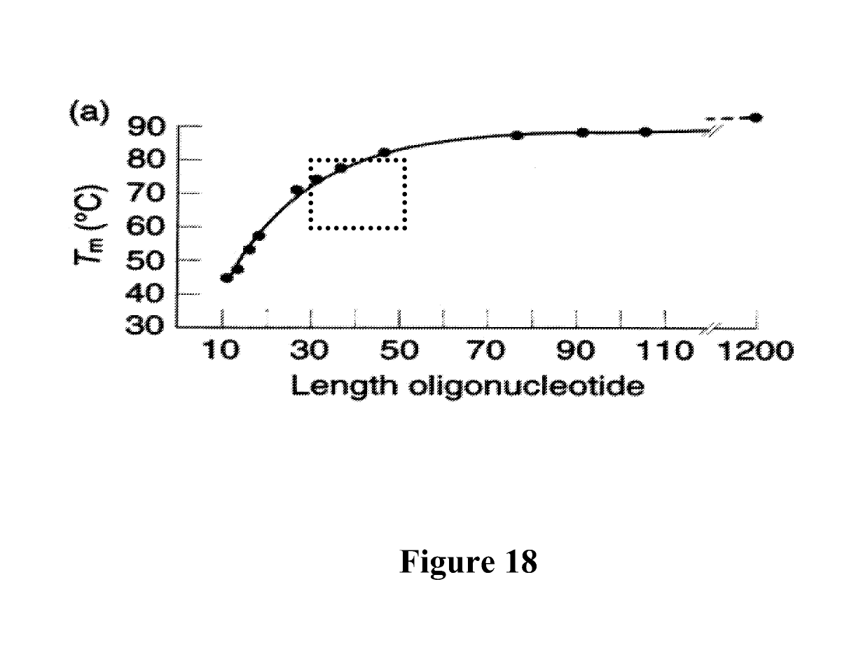

Primer design for selective amplification of low quantity mutant alleles should account for primer length, product length, and melting temperature. As seen in FIG. 13, a large number of designed PCR primers are 20 bp in length. Additionally, as seen in FIG. 14, PCR products commonly range in length from about 100-300 bp. Using these criteria, over one hundred 60-300 bp high stringency amplification products were selected through an automatic search using Oligo software, version 7 (Molecular Biology Insights, Inc., Cascade, Colo.). FIG. 15 shows that 68% of these products had a melting temperature (Tₘ) that was less than 85° C. while 90% of products had Tₘ less than 90° C. Using the same data, FIG. 16 exhibits a correlation between Tₘ and PCR product length and FIG. 18 shows the same phenomenon with a different data set. Therefore, Tₘ can be reduced by designing smaller PCR products. Another method of reducing Tₘ is to design PCR products with low guanine-cytosine (G-C) content (FIG. 17).

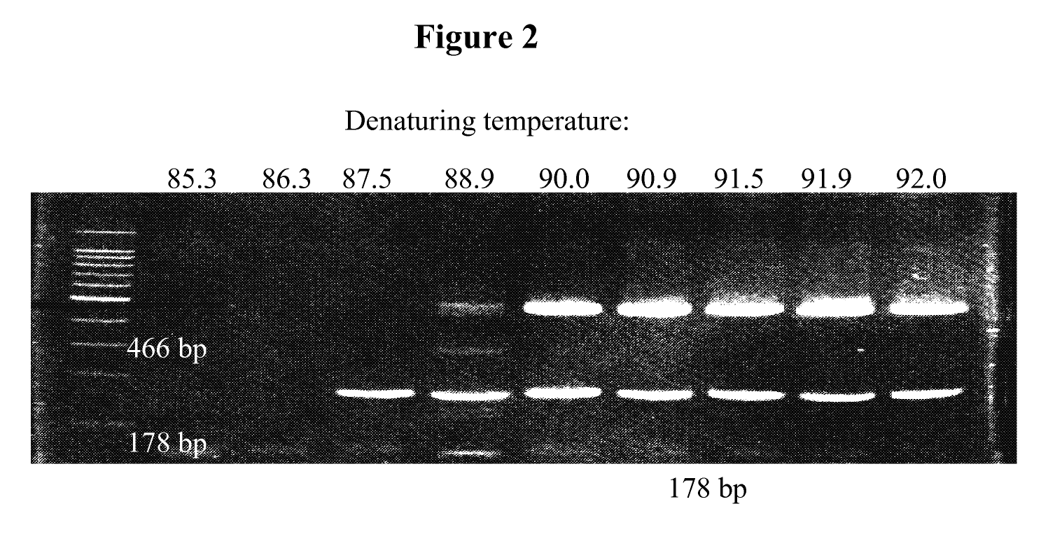

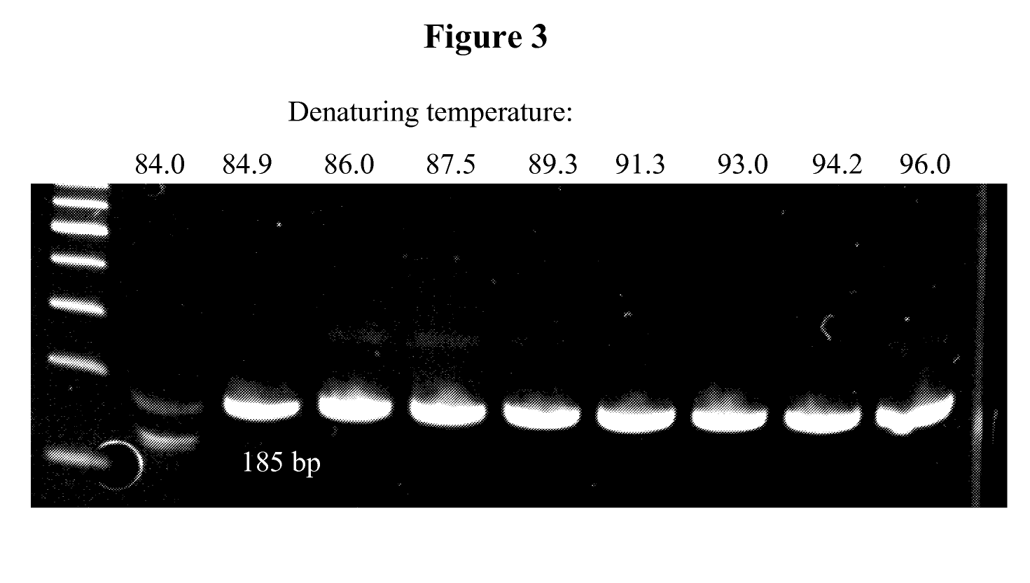

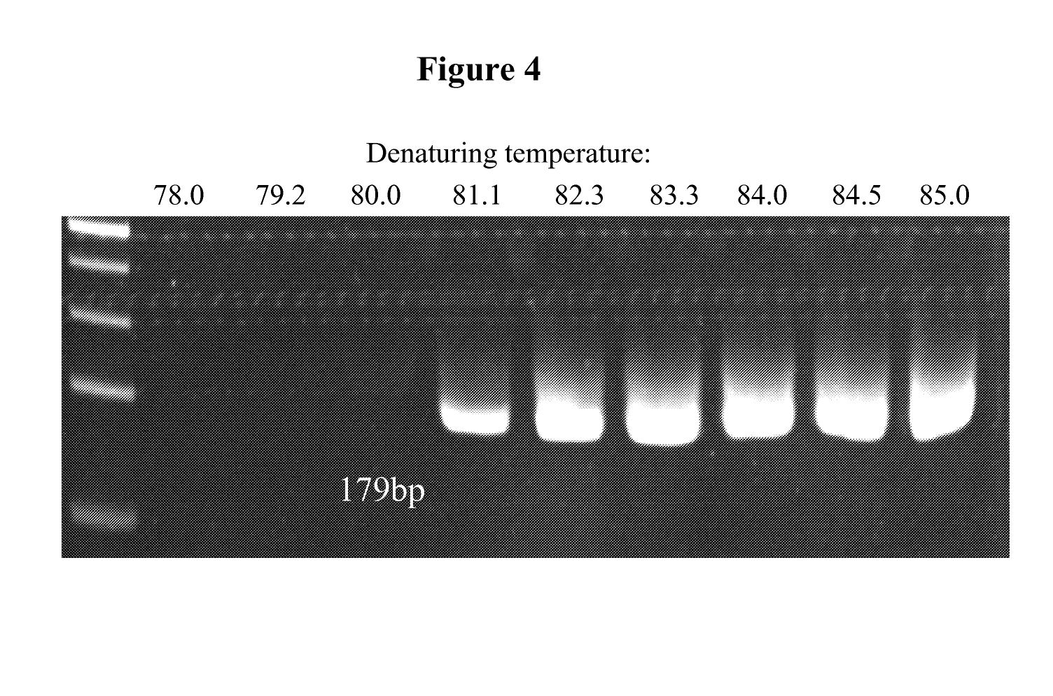

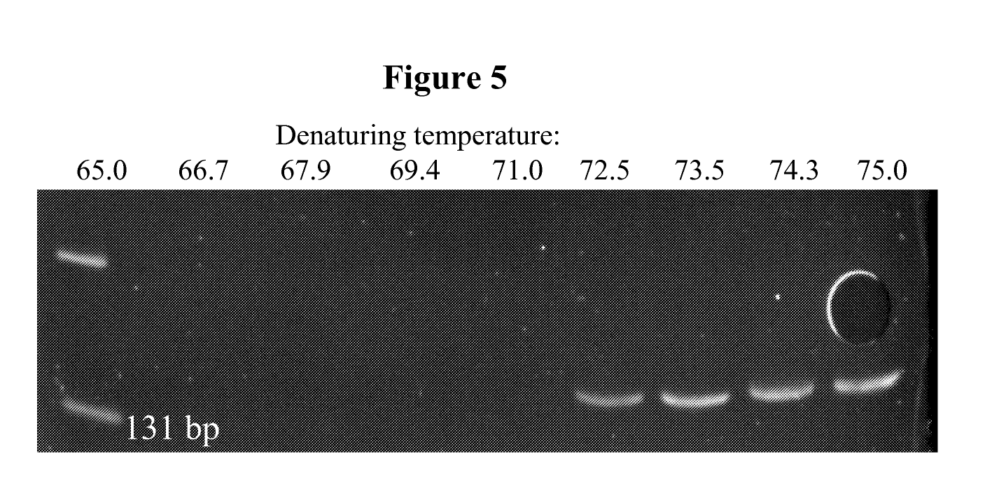

As seen in FIG. 2, a 466 bp fragment from mouse adenylate cyclase subtype 5 and a 178 bp fragment from glyceraldehyde-3-phosphate dehydrogenase (GAPDH) can be effectively amplified under denaturing temperatures of 90° C. and 87.5° C., respectively. Annealing and extension were performed at 72° C. for both reactions. Additionally, as seen in FIG. 3, a 185 bp fragment from human β-actin can be effectively amplified under a denaturing temperature of 85° C. with annealing and extension performed at 55° C. and 70° C., respectively. Furthermore, a 179 bp fragment from human β-actin can also be effectively amplified under a denaturing temperature of 81° C., with annealing and extension temperatures of 55° C. and 70° C., respectively. (FIG. 4). Finally, a 131 bp fragment from human chromosome 6 (AL731777/GI29801752) can be effectively amplified under a denaturing temperature of 72.5° C., with annealing and extension both performed at 60° C. (FIG. 5).

The denaturing temperatures may also be reduced using optimum co-solvents in the PCR reaction mix. For example, Tables 1 and 2 below (cited from Innis et al. ed. PCR Strategies, Academic Press, 1995) show the influence of some common co-solvents on the melting temperature (Tₘ) and strand dissociation temperature (Tₛₛ).

TABLE 1

| | | | | PCR | | | |
|---|---|---|---|---|---|---|---|
| | | | | Buffer: 5% formamide, | | | |
| | | PCR Buffer | | 10% glycerol | | | |
| DNA | % G + C | Tₘ | Tₛₛ | Tₘ | Tₛₛ | ΔTₘ | ΔTₛₛ |
| Lambda | 50 | 90.2 | 95 | 84 | 90-91 | −6.2 | −4-5 |
| Herring sperm | 42 | 86.5 | (98) | 82 | 86-92 | −4.5 | (−6) |
| Clostridium | 32 | 80.6 | 85 | 76 | 78-79 | −4.6 | −6 |
| Micrococcus | 72 | 99.5 | (102.5) | 93 | 94-97 | −6.5 | (−7) |
| luteus | | | | | | | |
| Human - HL60 | 42 | 86 | 96-98 | 80.6 | 87-92 | −5.4 | −6 |

( ): estimated value.

TABLE 2

| DNA | Tₘ (° C.) | ΔTₘ (° C.) |
|---|---|---|
| PCR Buffer Alone | 90.2 | |
| +10% Glycerol | 87.2 | −3.0 |
| +10% DMSO | 84.4 | −5.8 |
| +10% NMP | 79.6 | −10.6 |
| +10% Formamide | 83.6 | −6.6 |
| +5% Formamide and 10% Glycerol | 84.0 | −6.2 |

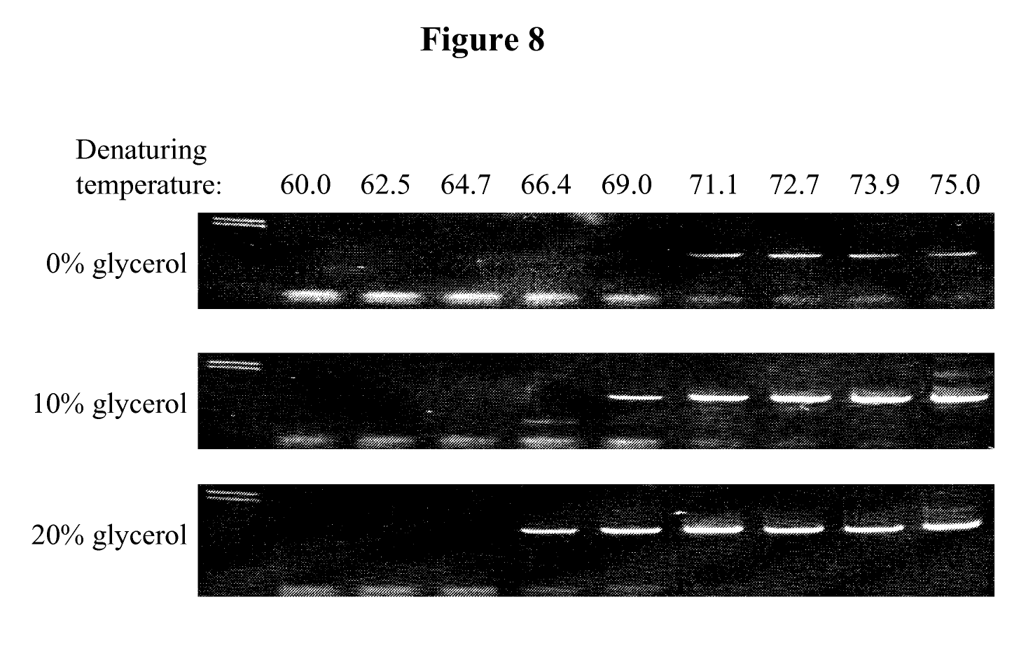

As seen in FIG. 8, a 131 bp fragment from human chromosome 6 can be effectively amplified under a denaturing temperature of 66.4° C. with 20% glycerol as a co-solvent. Annealing and extension temperatures were 35° C. and 60° C., respectively. Likewise, the same fragment can be effectively amplified under a denaturing temperature of 69° C. with 10% glycerol as a co-solvent. Annealing and extension temperatures were 35° C. and 60° C., respectively. These experiments show that PCR may be carried out at denaturing temperatures that are lower than the typical 94° C. or 95° C.

Lowering the Tₘ allows for the inclusion of restriction endonucleases in the PCR reaction mix without substantially irreversibly inactivate them. Several restriction endonucleases come from source organisms that grow at high temperatures (Table 3). Thus, they can withstand and even retain their endonuclease activity at high temperatures. Other available thermostable restriction endonucleases are shown in Tables 4 and 5. Furthermore, as little as eight thermostable restriction endonucleases can recognize nearly 60% of SNPs randomly generated from genes NM_{—}000015 to NM_{—}000034 (Table 6).

TABLE 3
Growth Temperatures for Microorganisms that Express Given
Thermostable Restriction Endonucleases

| | Growth Temperature of the |
|---|---|
| Restriction endonuclease | source microorganisms (° C.) |
| ApeKI | 95 |
| PspGI | 95 |
| PhoI | 98 |
| PabI | 103 |
| TmaI | 80 |
| EsaBC3I | 85 |
| EsaBC4I | 85 |
| MjaI | 85 |
| MjaII | 85 |
| MjaIII | 85 |
| MjaIV | 85 |
| MjaV | 85 |
| TceI | 85 |
| TliI | 85 |
| TneI | 85 |
| Tsp1I | 70 |
| Tsp32I | 70 |
| Tsp32II | 70 |
| Tsp45I | 70 |
| Tsp504I | 70 |
| Tsp505I | 70 |
| Tsp507I | 70 |
| Tsp509I | 70 |
| Tsp510I | 70 |
| Tsp514I | 70 |
| TspAI | 70 |
| MspNI | 75 |
| SuiI | 75 |
| TteI | 75 |
| Tth24I | 75 |
| Tth111I | 75 |
| Tth111II | 75 |
| TthHB8I | 75 |
| TthHB27I | 75 |
| TthRQI | 75 |
| TtmI | 75 |
| TtmII | 75 |
| TtrI | 75 |

TABLE 4
Thermostable Restriction Endonucleases (from New England Biolabs, Ipswich, MA)
and Their Compatibility with PCR Mixtures

| | | Optimum | | | | | |
|---|---|---|---|---|---|---|---|
| | Recognition | Temperature | Inactivated at | PCR | PCR | PCR | PCR |
| Enzyme | Sequence | (° C.) | 80° C./20 min | buffer 1 | buffer 2 | buffer 3 | buffer 4 |
| PspGI | CCWGG | 75 | n | +++ | +++ | +++ | +++ |
| PhoI | GGCC | 75 | n | <++ | ++ | +++ | +++ |
| MwoI | GC7NGC | 60 | Y | +++ | +++ | +++ | +++ |
| ApeKI | GCWGC | 75 | n | <++ | <++ | +++ | ++ |
| BsmI | CGAATCGCN | 65 | y | +++ | +++ | <+ | + |
| BsrI | ACTGGN | 65 | y | +++ | +++ | +++ | + |
| BstBI | TTCGAA | 65 | y | +++ | +++ | +++ | +++ |
| taqI | TCGA | 65 | y | +++ | +++ | +++ | +++ |
| TfiI | CAWTG | 65 | y | <++ | <++ | + | +++ |
| TspRI | NNCASTGNN | 65 | n | + | + | <+ | ++ |
| TseI | GCWGC | 65 | n | +++ | +++ | +++ | +++ |
| Tsp509I | AATT | 65 | n | +++ | +++ | +++ | +++ |
| Tsp45I | GTSAC | 65 | n | <+ | +++ | − | ++ |

TABLE 5
Restriction Enzymes That Do Not Inactivate When Exposed To 80° C.
for 20 Minutes (from New England Biolabs, Ipswich, MA)

| Enzyme | Optimum Temperature (° C.) |
|---|---|
| BclI | 50 |
| BgLII | 37 |
| BlpI | 37 |
| BsaXI | 37 |
| BsoBI | 37 |
| BsR FI | 37 |
| BstBI | 65 |
| BstEII | 60 |
| BstNI | 60 |
| BstUI | 60 |
| BstZ17I | 37 |
| CspCI | 37 |
| MwoI | 60 |
| PhoI | 75 |
| PspGI | 75 |
| SfiI | 50 |
| SmlI | 55 |
| TfiL | 75 |
| TliI | 75 |
| TseI | 65 |
| Tsp45I | 65 |
| Tsp509I | 65 |
| TspRI | 65 |
| Tth111I | 65 |

TABLE 6
Percentages of SNP Sequences Recognized by
Eight Selected Thermo-Tolerant Restriction
Endonucleases (100 Randomly Picked SNPs
from Genes NM_000015-NM_000034)

| | Recognition | |
|---|---|---|
| Enzyme | Sequence | % |
| BseLI | CCNNNNNNGG | 31 |
| MwoI | GCNNNNNNNGC | 18 |
| BsaXI | ACNNNNNCTTC | 12 |
| TaqI | TCGA | 4 |
| TasI | AATT | 4 |
| BstHHI | GCGC | 6 |
| TspRI | CASTG | 4 |
| Tsp45C | GTSAC | 4 |

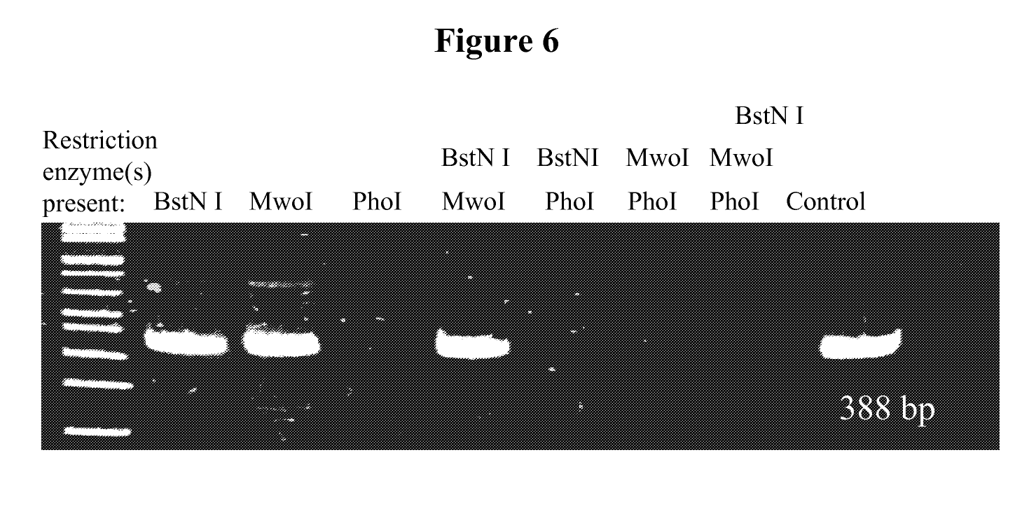

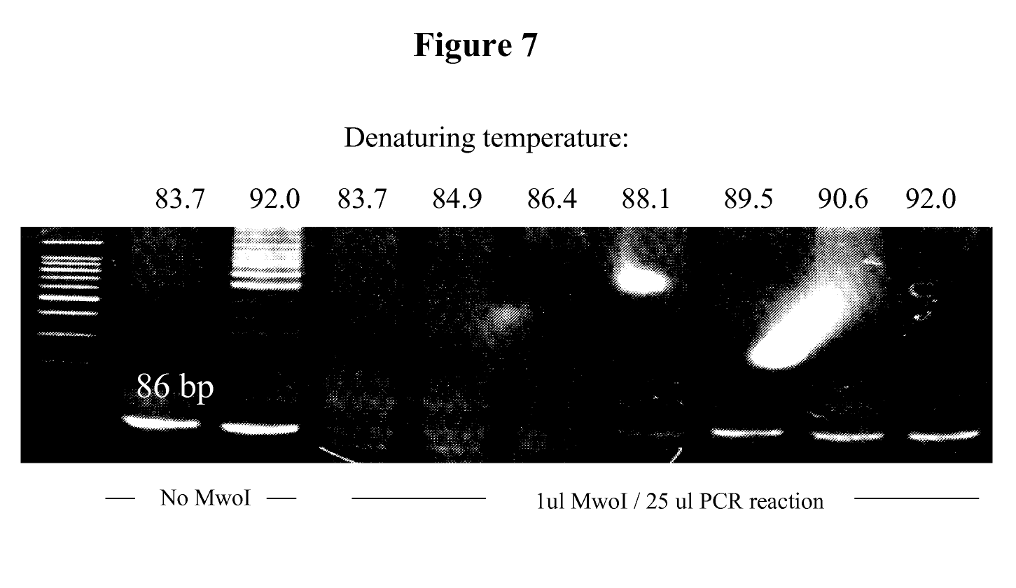

As seen in FIG. 6, the restriction endonuclease PhoI retains activity under a 92° C. denaturing temperature and blocks the amplification of a 388 bp fragment from human GAPDH containing PhoI, MwoI, and BstNI restriction sites. The fragment was amplified using brain cDNA by Advantage 2 PCR kit (Clontech, Mountain View, Calif.). Denaturing, annealing, and extension temperatures were 92° C., 60° C., and 75° C., respectively. In FIG. 7, MwoI retains activity under an 83° C.-88° C. denaturing temperature and effectively blocks the amplification of an 86 bp fragment from human guanylate cyclase 2F containing an MwoI restriction site. Annealing and extension temperatures were 60° C. and 70° C., respectively.

Because the denaturing temperature may be lowered, thermostable restriction endonucleases that are active during PCR are widely available, and because the large number of SNP wild type gene sequences that are recognized by just a small number of thermostable restriction endonucleases, this strategy of selective amplification of low-quantity mutant alleles by inclusion of restriction endonucleases in a one-pot PCR reaction should be widely applicable in most circumstances.

### Example 2

### Selective Amplification of Low-Quantity Mutant Alleles by Inclusion of Primers Designed to Induce Hairpin Formation in Wild-Type Amplicons

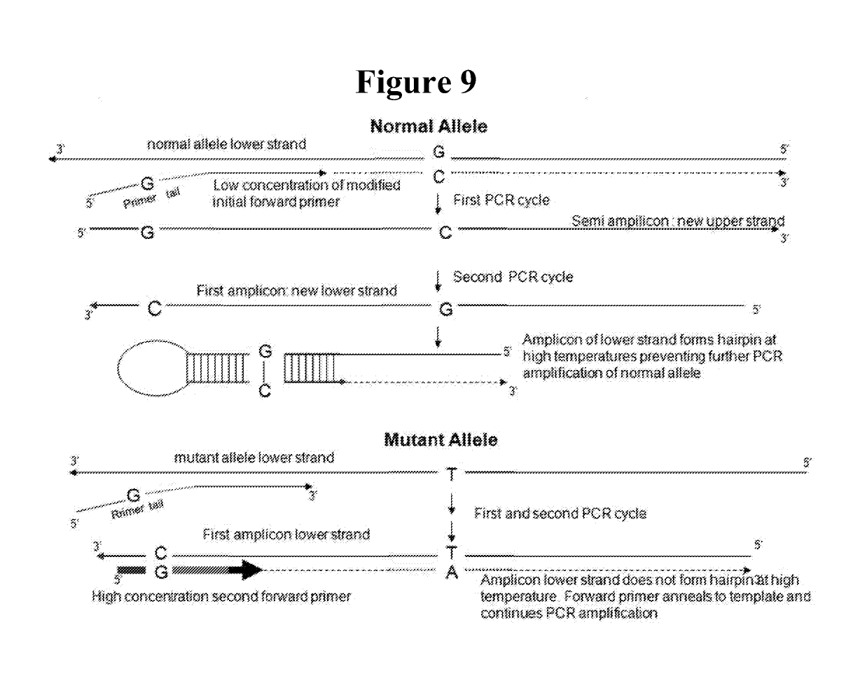

As illustrated in FIG. 9, to selectively amplify a mutant gene allele, another strategy includes (1) inclusion of a low concentration of a specific forward primer that produces a hairpin-forming amplicon of the normal allele after the first round of PCR that cannot serve as a template for subsequent amplification; and (2) inclusion of a high concentration of a second forward primer with an appropriate Tₘ that anneals only to amplification products resulting from extension of the initial forward primer and not to the starting DNA, as well as a reverse primer, allowing for selective PCR amplification of the mutant allele.

Experiments have been performed for this strategy using a single nucleotide base difference between the rat (as mutant) (SEQ ID NO: 47) and mouse (as wild) β-actin gene (SEQ ID NO: 48). Mouse and rat brain total RNA were isolated using TRIzol reagent (Ambion/Life technologies, Grand Island, N.Y.). 1 μg total RNA was used for cDNA synthesis with an Advantage RT-for PCR kit (Clontech, Mountain View, Calif.) using an oligo(dT) primer according to the manufacturer's protocol and diluted to 100 μl. Rat cDNA was diluted 10, 50, 100, 200, 500, 1000, 2000 and 5000 fold, and mixed 1:1 with undiluted mouse cDNA. PCR# was performed using an Advantage 2 PCR kit (Clontech, Mountain View, Calif.). Each 20 μl reaction contained a 2 μl mouse and rat cDNA mixture. The final concentration of initial forward primer (SEQ ID NO: 44), successive forward primer (SEQ ID NO: 45), and reverse primer (SEQ. ID NO: 46) were 0.008 μM, 8 μM, and 1 μM, respectively. The first cycle of the PCR was carried out at 94° C. for 1 minute, 89° C. for 15 seconds, and 70° C. for 10 minutes. Then, PCR was carried out at 94° C. for 1 minute, 89° C. for 15 seconds, and 70° C. for 1 minute for 36 cycles.

Next, a 15 μl digestion reaction containing 7 μl of the above PCR product and 0.750 XbaI (20 U/μl, New England Biolabs, Ipswich, Mass.) was incubated at 37° C. for one hour. Alternatively, the digestion reaction containing 7 μl of the above PCR product and 0.50 μl Tru9I (12 U/μl, Promega, Madison, Wash.) was incubated at 65° C. for one hour.

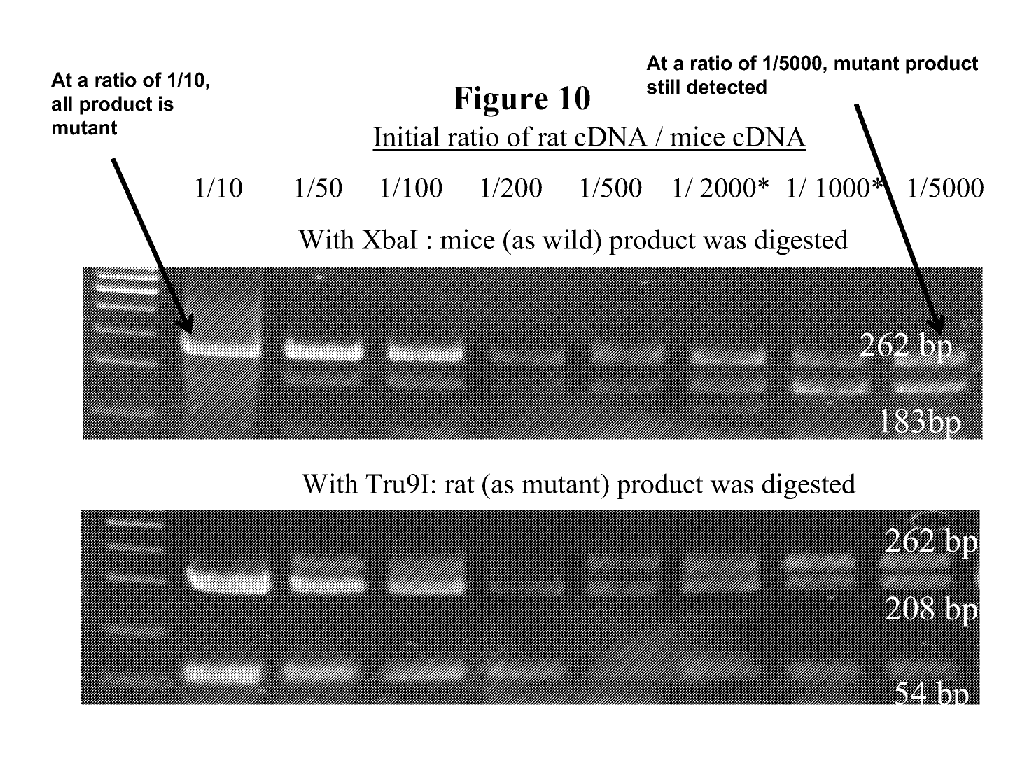

The results of this experiment (FIG. 10) showed that this method allowed the detection of the rat allele even when its starting DNA copies represented only 0.02% of the mouse allele.

### Example 3

### Selective Amplification of Mutant Alleles by Addition of Thermostable Restriction Endonucleases

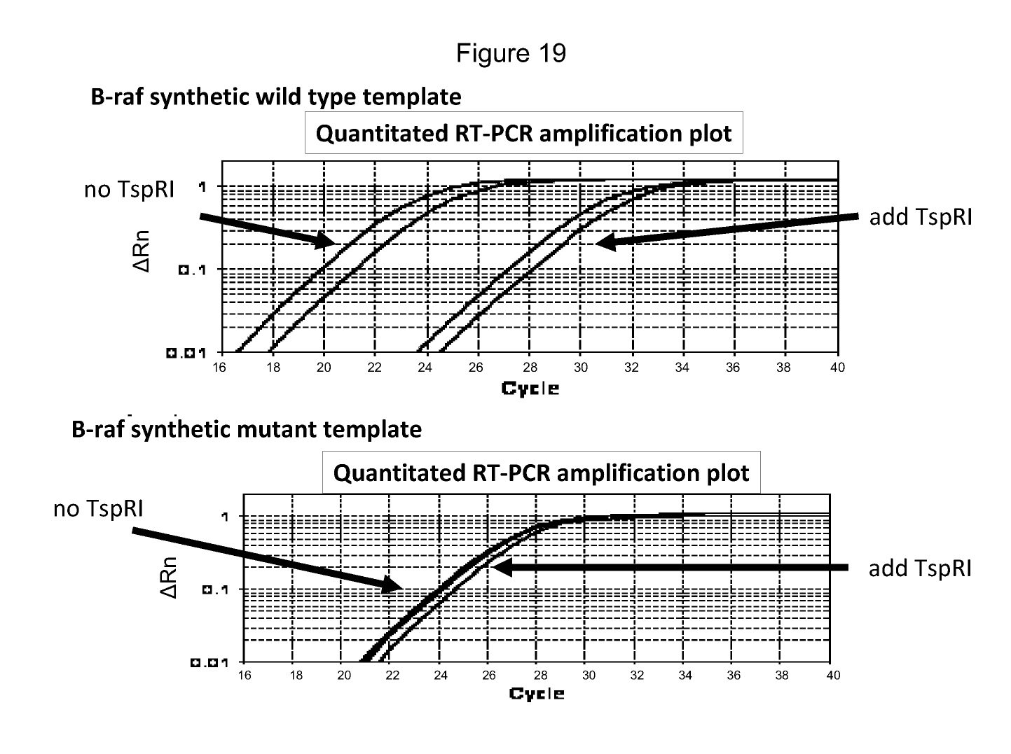

Quantitative polymerase chain reaction (qPCR) was performed on an Applied Biosystems 7500 PCR system using version 2.06 software (Applied Biosystems/Life technologies, Carlsbad, Calif.). Two different qPCR kits were used: SYBR Advantage qPCR premix (Clontech Laboratories, Inc., Mountain View, Calif.) and Power SYBR Green PCR Master Mix (Applied Biosystems). qPCR conditions followed the manufacturers' recommendations. Reactions were carried out in 20 μl reaction volumes and included 0 (control) or 2-3 units of the restriction endonuclease as indicated in the FIGS. 19-22. In studies in which the wildtype template was degraded before qPCR, Applied Biosystems' kit was used. PCR was initiated at the temperature that was optimum for the restriction enzyme in use for 10 to 15 minutes. The temperature was then increased to 95° C. for 10 minutes and was followed by 40 cycles of denaturation temperatures of 84° C.-87° C. and annealing/extension temperatures of 65° C. for 1 to 2 minutes. In studies in which both the wildtype template and the amplification product were degraded during qPCR, Clontech's qPCR kit was used. PCR was initiated at 85° C. for 2 minutes to denature the template. This was followed by 40 cycles of PCR that included a denaturation step at 85° C. for 15 seconds and annealing/extension and degrading wild type product at 65° C. for 1 to 2 minutes or 65° C. for 1 minute and increasing to 75° C. for another 1 minute. PCR products were purified using the QAquick PCR purification kit and sequenced by the Sanger sequence method (Genewiz, N.J.). The sequencing results were analyzed by Chromas Lite software.

FIG. 19 shows the amplification plots of synthetic B-raf templates representing wild type (SEQ ID NO:67) or mutant (SEQ ID NO:68) sequences of the B-raf gene in the absence or presence of the thermostable enzyme TspRI. Quantitative PCR was performed on a synthetic wild type B-raf sequence (containing a TspRI endonuclease recognition site) or a synthetic mutant B-raf sequence (lacking a site, i.e., the nucleotide sequence of the recognition site has been changed by at least one nucleotide compared to the wild type sequence). PCR was performed at a reduced denaturation temperature of 85° C. for 40 cycles. The primer used for the reaction is listed in Table 7 below. Note that the TspRI enzyme destroys products of the wild type template during the PCR reaction (top tracing) resulting in a rightward shift in the amplification plot and Ct value indicative of a >99% enzymatic destruction in the PCR product of the wild type allele. This would allow for an enrichment of the amplification of a less abundant mutant allele.

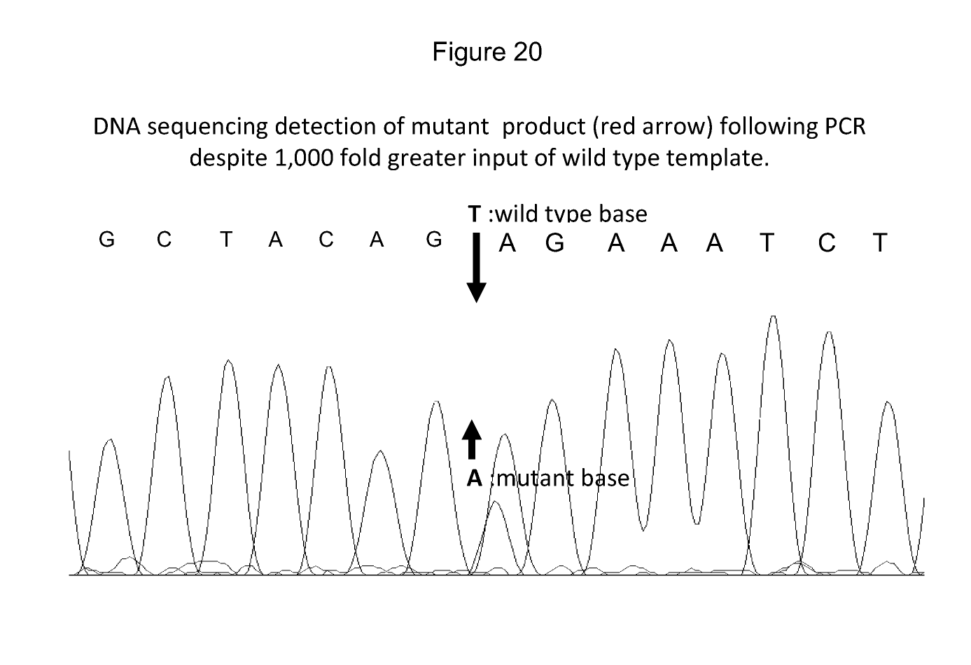

FIG. 20 shows the DNA sequencing results of PCR products following amplification of a wild type and a mutant template of the B-raf gene present in a 1,000:1 ratio. PCR was performed with a 1,000-fold excess of wild type to mutant template co-incubated in the presence of the thermostable endonuclease TspRI which selectively destroys products of the wild type template. Note that despite a 1,000-fold greater amount of wild type template, the product of the mutant allele (bottom arrow) was still detectable following PCR.

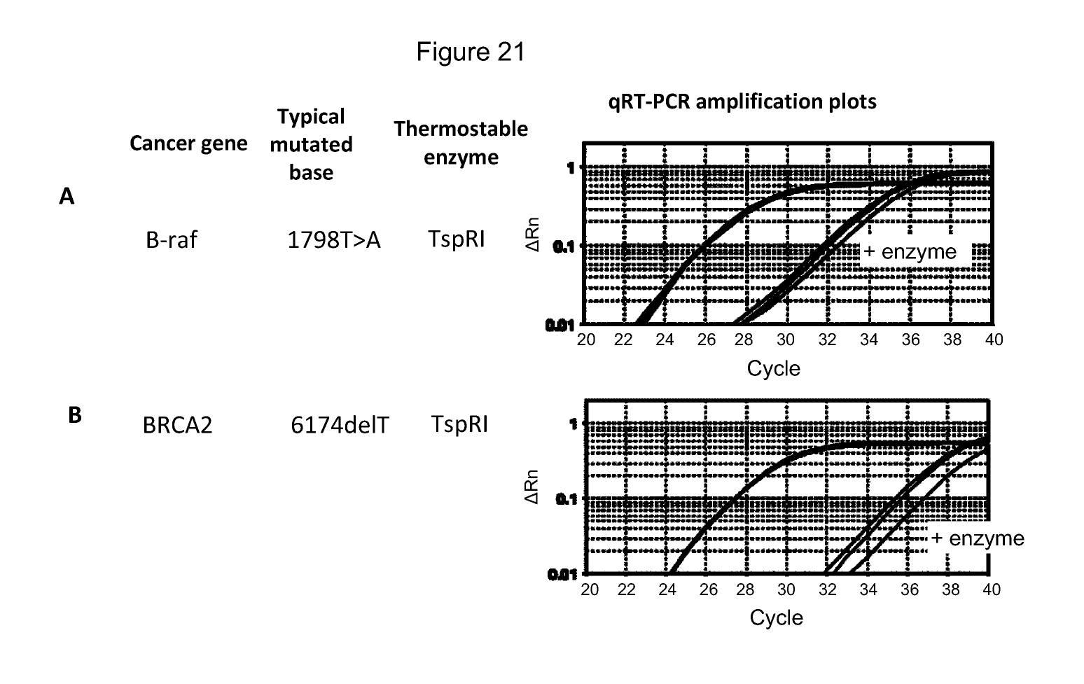

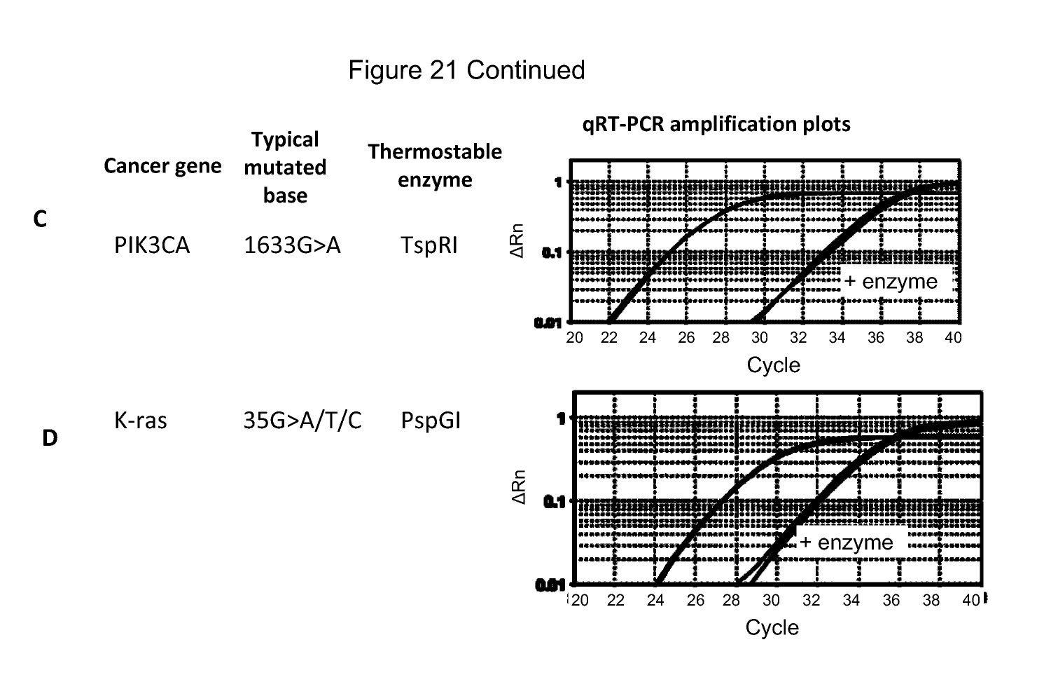

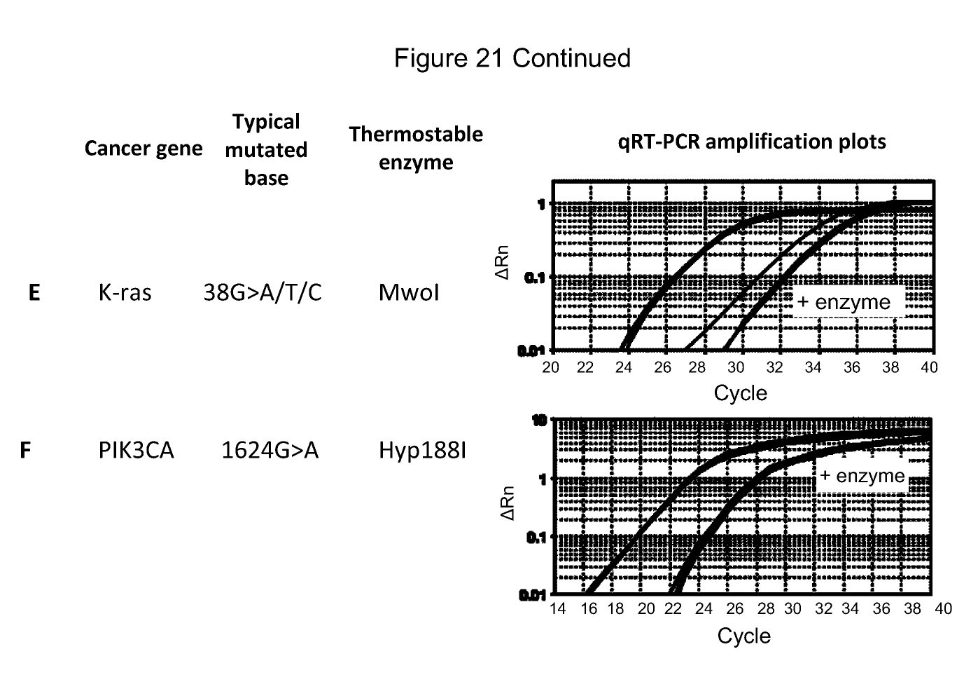

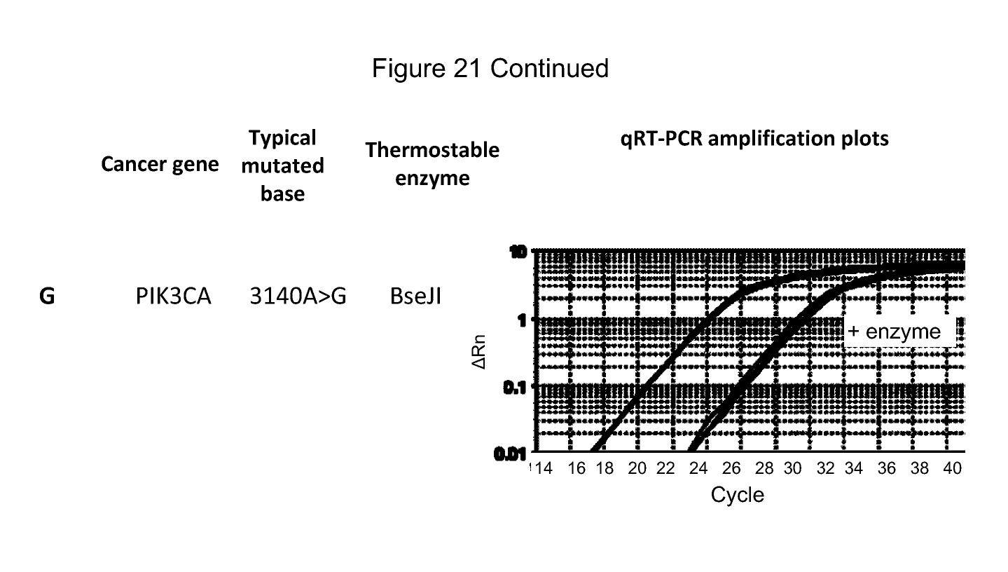

FIGS. 21A-21G show the amplification plots of wild type genomic DNA in the absence or presence of thermostable restriction endonucleases known to digest wild type regions of several genes that commonly encode mutations in solid tumors. This feasibility study demonstrates that these enzymes degrade PCR products of wild type alleles which would allow for enhanced PCR amplification of mutant alleles which lack recognition sites for these enzymes.

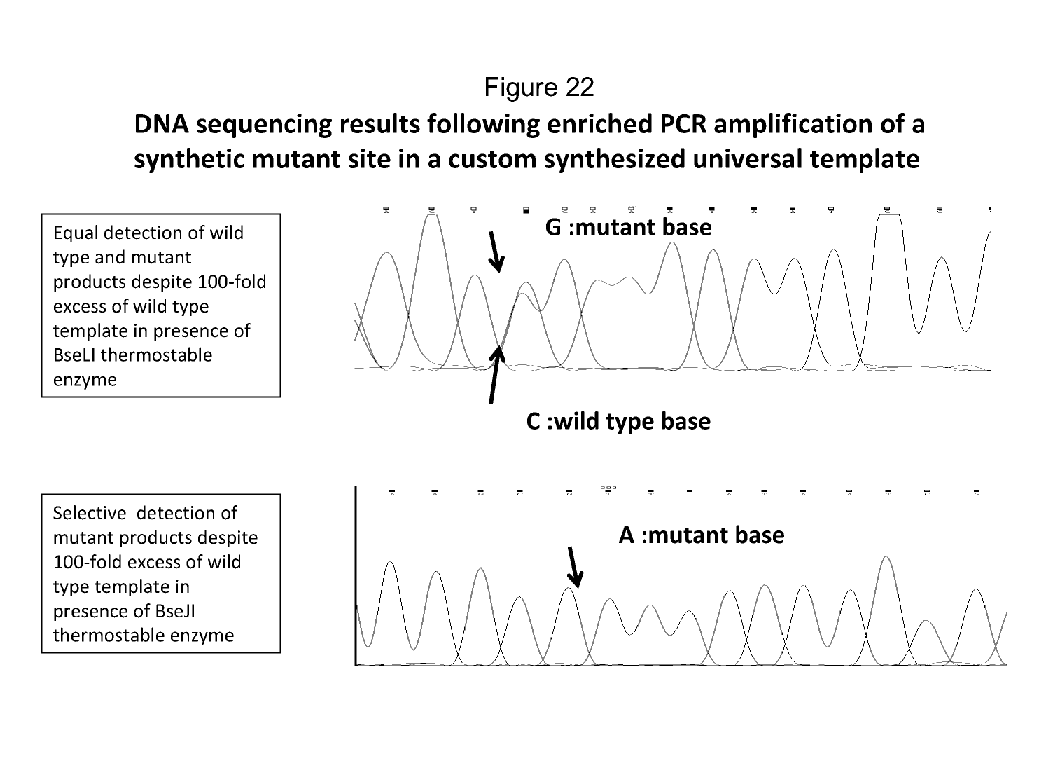

FIG. 22 shows the DNA sequencing results following enriched PCR amplification of a synthetic mutant site in a set of custom synthesized universal templates (SEQ ID NO:69-70), which allows for the identification of optimal conditions for the activities of thermostable enzymes. These enzymes would be used to degrade products of wild type amplicons allowing for the selective amplification of mutant templates present in low abundance in solid tumors. A custom synthetic DNA template containing enzyme recognition sites for 30 thermostable endonucleases was designed (“wild type template”, SEQ ID NO:69) using the following selection criteria: (1) the enzyme can survive in environments that are greater than 80° C. for at least 20 minutes; and (2) the organism from which the enzyme originates grows in environments having a temperature between 50-70° C. These enzyme sites would be present in wild type genes but absent in mutant alleles. A second synthetic universal DNA template containing the complimentary mutations was synthesized (“mutant template”, SEQ ID NO:70). Known ratios of this wild type and mutant template were combined in the presence of selected thermostable enzymes. Examples of two experiments are presented; a mixture of a wild type universal template and a mutant universal template were mixed in a 100:1 ratio and subjected to PCR amplification at a reduced denaturation temperature (85° C.) in the presence of BseLI (top tracing) and Bse JI (bottom tracing). These enzymes successfully degraded the wild type amplification products allowing for the equal detection of the mutant product (top tracing) or the detection of only the mutant product (bottom tracing).

The primers used in the experimental results shown in FIGS. 19-22 are as follows:

TABLE 7
List of Primers

| | Accession | |
|---|---|---|
| Primer for | number | Primer sequence (5′ to 3′) |
| Figure 19, B- | | CTTATTGACTCTAAGAGGAAAGATG |
| raf synthetic | | AAGTACTATG (SEQ ID NO: 51) |
| (wild/mutant) | | CAATTCTTACCATCCACAAAATGGAT |
| template | | CCAG (SEQ ID NO: 52) |
| Figure 21A | NM_004333 | CTTATTGACTCTAAGAGGAAAGATGA |
| B-raf | | AGTACTATG (SEQ ID NO: 53) |
| | | CAATTCTTACCATCCACAAAATGGAT |
| | | CCAG (SEQ ID NO: 54) |
| Figure 21B | NM_000059 | CTGCAAATACTTGTGGGATTTTTAGC |
| BRCA2 | | ACAGCCAG (SEQ ID NO: 55)* |
| | | GACTTGCTTGGTACTATCTTCTATTT |
| | | CAGAAAACA (SEQ ID NO: 56) |
| Figures 21C | NM_006218 | GAGACAATGAATTAAGGGAAAATGAC |
| and 21F | | AA (SEQ ID NO: 57) |
| PIK3CA | | CTGTGACTCCATAGAAAATCTTTCTC |
| | | (SEQ ID NO: 58) |
| Figure 21D | NM_033360 | GCTGAAAATGACTGAATATAAACTTG |
| K-ras | | TGGTAGTTGGAACTG |
| | | (SEQ ID NO: 59)* |
| | | ATATTCGTCCACAAAATGATTCTGAA |
| | | TTAGCTGTA (SEQ ID NO: 60) |
| Figure 21E | NM_033360 | GACTGAATATAAACTTGTGGTAGTTG |
| K-ras | | GCGCTGGTG (SEQ ID NO: 61)* |
| | | AGAGAAACCTTTATCTGTATCAAAGA |
| | | ATGGTCCTG (SEQ ID NO: 62) |
| Figure 21G | NM_006218 | AGACCCTAGCCTTAGATAAAACTGAG |
| PIK3CA | | CAAG (SEQ ID NO: 63) |
| | | TCAATGCATGCTGTTTAATTGTGTGG |
| | | AAGA (SEQ ID NO: 64) |
| Figure 22 | | TTGACTCTATGATGAAAGATGTTGTT |
| For | | (SEQ ID NO: 65) |
| universal | | ATTACGATCCACAAAATGTATGCAG |
| template | | (SEQ ID NO: 66) |

*Mismatch to construct thermo-stable restriction endonuclease recognition site

The universal wildtype template contains 30 thermostable restriction sites, which sites are not in the universal mutant template. These sites are listed in Table 8 below.

TABLE 8
Thermostable Restriction Sites in the
Universal Wildtype Template

| | | | | average | |
|---|---|---|---|---|---|
| | | | | fragment | |
| | | | | length | |
| | | | | for | |
| | | | | enzyme | |
| | | | | over | |
| recog- | | Optimum | Growth | entire | Commercial |
| nition | | temper- | Temper- | human | Availa- |
| sequence | Name | ature | ature | genome | bility |
| AATT | TasI | 65 | 72 | 133 | Fermentas |
| TTAA | Tru1I | 65 | 60 | 150 | Fermentas |
| ATGAA | TspDTI | 70 | 70 | 276 | Eurx |
| CCWGG | BstNI | 60 | 55 | 292 | NEB |
| CCWGG | PspGI | 75 | 95 | 292 | NEB |
| CASTG | TscAI | 65 | 70 | 330 | Fermentas |
| CASTG | TspRI | 65 | 70 | 330 | NEB |
| GGCC | PhoI | 75 | 98 | 343 | NEB |
| ACNGT | TaaI | 65 | 72 | 380 | Fermentas |
| CCNNNN | BseLI | 55 | 55 | 382 | Fermentas |
| NNNGG | | | | | |
| GCNNNN | MwoI | 60 | 60 | 469 | NEB |
| NNNGC | | | | | |
| GCWGC | TseI | 65 | 70 | 556 | NEB |
| GCWGC | ApeKI | 75 | 95 | 556 | NEB |
| GGATG | BtsCI | 50 | 55 | 558 | NEB |
| GAWTC | TfiI | 65 | 70 | 610 | NEB |
| GTSAC | Tsp45I | 65 | 70 | 726 | NEB |
| CTRYAG | BstSF1 | 60 | 55 | 827 | SibEnzymes |
| CTYRAG | SmlI | 55 | 30 | 1059 | NEB |
| WGTACW | TatI | 65 | 65 | 1063 | Fermentas |
| ACGT | TaiI | 65 | 65 | 1330 | Fermentas |
| GWGCWC | BsiHkAI | 65 | 37 | 1406 | NEB |
| GKGCMC | BseSI | 55 | 55 | 1444 | Fermentas |
| GCGC | BstHHI | 50 | 55 | 1728 | SibEnzymes |
| TCGA | TaqI | 65 | 70 | 1894 | Fermentas |
| ACGGA | TspGWI | 70 | 70 | 2878 | Eurx |
| GCSGC | TauI | 55 | 70 | 3188 | Fermentas |
| TGATCA | BclI | 50 | 70 | 3911 | NEB |
| CGCG | BstUI | 60 | 55 | 3941 | NEB |
| GACNNN | Tth111I | 65 | 75 | 4068 | NEB |
| GTC | | | | | |
| GATNNN | BseJI | 65 | 65 | 6436 | Fermentas |
| NATC | | | | | |

### Example 4

### Cancer Detection by Selective Amplification of Mutant Alleles

Additional PCR conditions (primer design, denaturation temperatures, choice of thermostable restriction endonucleases) for both of the strategies set forth in the above Examples have been determined herein for the most common single nucleotide point or small insertion/deletion mutations in breast cancer (e.g. BRCA1, BRCA2, K-ras, B-raf, PIK3CA and EGFR) and other solid tumor cancers. The PCR conditions are shown in FIGS. 11 and 12.

The methods disclosed herein enhance the sensitivity of detecting cancer in tissue samples. The methods modify existing PCR methods to detect, with greater sensitivity, the existence of, e.g., a single nucleotide point mutation known to cause a variety of solid tumors (breast, colon, lung cancer).

The methods disclosed herein offer much greater sensitivity for detection of cancer when only a small fraction of the cells have the mutation. Current PCR methods can only amplify a mutant allele from a mixture of mutant and normal cells when the mutant allele represents at least about 10% of the total cells. The methods of the present invention enhance this sensitivity as much as 500-fold such that mutant cells may be detected when they represent only 0.02% of the total cells.

All documents cited in this application are hereby incorporated by reference as if recited in full herein.

Although illustrative embodiments of the present invention have been described herein, it should be understood that the invention is not limited to those described, and that various other changes or modifications may be made by one skilled in the art without departing from the scope or spirit of the invention.

What is claimed is:

1. A method for selectively amplifying a mutant allele comprising:
(a) contacting a sample comprising a mutant and a normal allele of a gene with at least one primer that selectively modifies the normal allele of the gene and causes further amplification of the normal allele to fail or to be substantially reduced; and
(b) selectively amplifying the mutant allele of the gene or a portion thereof.

2. The method according to claim 1, which is carried out with a polymerase chain reaction (PCR), which reaction includes denaturing, extension, and annealing steps.

3. The method according to claim 2, wherein the primer introduces a thermostable restriction endonuclease site into an amplicon of the normal allele but not an amplicon of the mutant allele after two rounds of PCR, and wherein step (b) comprises contacting the sample with a thermostable restriction endonuclease that recognizes the site introduced into the amplicon of the normal allele.

4. The method according to claim 3, wherein the thermostable restriction endonuclease is active in a PCR reaction mix.

5. The method according to claim 3, wherein the denaturing step of the PCR is carried out at a temperature that does not substantially irreversibly inactivate the thermostable restriction endonuclease.

6. The method according to claim 5, wherein the denaturing step of the PCR is carried out at a temperature between about 70° C. and about 90° C.

7. The method according to claim 3, wherein the annealing step and/or the extension step of the PCR are carried out at a temperature at which the thermostable restriction endonuclease is active.

8. The method according to claim 7, wherein the annealing step of the PCR is carried out at a temperature between about 55° C. and about 72° C.

9. The method according to claim 7, wherein the extension step of the PCR is carried out at a temperature between about 60° C. and about 72° C.

10. The method according to claim 3, wherein the thermostable restriction endonuclease is selected from the group consisting of ApeKI, BclI, BgLII, BlpI, BsaXI, BseJI, BseLI, BseSI, BsiHkAI, BsmI, BsoBI, BsR FI, BsrI, BstBI, BstEII, BstHHI, BstNI, BstSF1, BstUI, BstZ17I, BtsCI, CspCI, EsaBC3I, EsaBC4I, Hyp188I, MjaI, MjaII, MjaIII, MjaIV, MjaV, MspNI, MwoI, PabI, PhoI, PspGI, SfiI, SmlI, SmlI, SuiI, TaaI, TaiI, TaqI, TasI, TatI, TauI, TceI, TfiI, TfiL, TliI, TmaI, TneI, Tru1I, TseI, Tsp1I, Tsp32I, Tsp321I, Tsp45C, Tsp45I, Tsp504I, Tsp505I, Tsp507I, Tsp509I, Tsp510I, Tsp514I, Tsp560I, TspAI, TspDTI, TspGWI, TspRI, TteI, Tth111I, Tth111II, Tth24I, TthHB27I, TthHB8I, TthRQI, TtmI, TtmII, and TtrI.

11. The method according to claim 10, wherein the thermostable restriction endonuclease is selected from the group consisting of ApeKI, BsaXI, BseJI, BseLI, BsmI, BsrI, BstBI, BstHHI, Hyp188I, MwoI, PhoI, PspGI, TaqI, TasI, TfiI, TseI, Tsp45C, Tsp45I, Tsp509I, and TspRI.

12. The method according to claim 1, wherein the amount of the mutant allele is at least about 0.02% of the normal allele in the sample.

13. The method according to claim 1, wherein the mutant allele is a marker for a cancer.

14. The method according to claim 1, wherein the mutant allele is selected from the group consisting of 185 deletion AG in BRCA1, 5382 insertion C in BRCA1, 6174 deletion T (6174 del T) in BRCA2, G12D 35G>A/T/C in K-ras, 38G>A/T/C in K-ras, V600E 1798 T>A in B-raf, E545K 1633 G>A in PIK3CA, 1624G>A in PIK3CA, 3140A>G in PIK3C1, and L858R 2573 T>G in EGFR.

15. The method according to claim 1, wherein a first forward primer produces a first and a second amplicon after two rounds of amplification, the first amplicon being an amplicon of the normal allele, which cannot serve as a template for subsequent amplification, and the second amplicon being an amplicon of the mutant allele.

16. The method according to claim 15, further comprising contacting the sample with a second forward primer that anneals to the second amplicon.

17. The method according to claim 16, wherein the ratio of the first forward primer to the second forward primer is less than 1:10.

18. The method according to claim 15, wherein the first amplicon forms a hairpin.

19. The method according to claim 15, wherein the first forward primer is selected from the group consisting of TCTGTCCTGGGATTCTCTTGAGATGTGGTCAATGGAAGAAACCACCAAG (SEQ ID NO: 29), GGGACACTCTAAGATTTTCTTCGCGTTGAAGAAGTACAAAATGTCATTA (SEQ ID NO: 34), and GACAGATTTTCCACTTGCTGTGCGGAAGCTTCATAAGTCAGTCTCATCTGCAAA TAC (SEQ ID NO: 39); and the second forward primer is selected from the group consisting of CCTGGGATTCTCTTGAGATGTGGTCAAT (SEQ ID NO: 30), ACACTCTAAGATTTTCTTCGCGTT (SEQ ID NO: 35), and ATTTTCCACTTGCTGTGCGGAAGCTTCATA (SEQ ID NO: 40), respectively.

20. The method according to claim 15, wherein the mutant allele is selected from the group consisting of 5382 insertion C in BRCA1, 185 deletion AG in BRCA1, and 6174 del T in BRCA2.

21. A method for detecting a mutant allele of a gene, if present, in a sample comprising:
(a) contacting the sample with a thermostable restriction endonuclease that recognizes a sequence in a normal allele of the gene but not in a mutant allele of the gene;
(b) amplifying the mutant allele of the gene or a portion thereof, if present in the sample; and
(c) before or during step (b), selectively degrading the normal allele of the gene.

22. The method according to claim 21, wherein step (b) comprises using a polymerase chain reaction (PCR), which reaction includes denaturing, extension, and annealing steps.

23. The method according to claim 21, wherein the amount of the mutant allele is at least about 0.02% of the normal allele in the sample.

24. The method according to claim 21, wherein the thermostable restriction endonuclease is selected from the group consisting of ApeKI, BclI, BgLII, BlpI, BsaXI, BseJI, BseLI, BseSI, BsiHkAI, BsmI, BsoBI, BsR FI, BsrI, BstBI, BstEII, BstHHI, BstNI, BstSF1, BstUI, BstZ17I, BtsCI, CspCI, EsaBC3I, EsaBC4I, Hyp188I, MjaI, MjaII, MjaIII, MjaIV, MjaV, MspNI, MwoI, PabI, PhoI, PspGI, SfiI, SmlI, SmlI, SuiI, TaaI, TaiI, TaqI, TasI, TatI, TauI, TceI, TfiI, TfiL, TliI, TmaI, TneI, Tru1I, TscAI, TseI, Tsp1I, Tsp32I, Tsp32II, Tsp45C, Tsp45I, Tsp504I, Tsp505I, Tsp507I, Tsp509I, Tsp510I, Tsp514I, Tsp560I, TspAI, TspDTI, TspGWI, TspRI, TteI, Tth111I, Tth111II, Tth24I, TthHB27I, TthHB8I, TthRQI, TtmI, TtmII, and TtrI.

25. The method according to claim 24, wherein the thermostable restriction endonuclease is selected from the group consisting of ApeKI, BsaXI, BseJI, BseLI, BsmI, BsrI, BstBI, BstHHI, Hyp188I, MwoI, PhoI, PspGI, TaqI, TasI, TfiI, TseI, Tsp45C, Tsp45I, Tsp509I, and TspRI.

26. The method according to claim 21, wherein the sequence in the normal allele recognized by the thermostable restriction endonuclease is natively present in the normal allele.

27. The method according to claim 21, wherein the sequence in the normal allele recognized by the thermostable restriction endonuclease is introduced by a primer used to amplify the gene or a portion thereof.

28. The method according to claim 21, wherein the mutant allele of the gene is a marker for a cancer.

29. The method according to claim 21, wherein the mutant allele is selected from the group consisting of 185 deletion AG in BRCA1, 5382 insertion C in BRCA1, 6174 del T in BRCA2, G12D 35G>A/T/C in K-ras, 38G>A/T/C in K-ras, V600E 1798 T>A in B-raf, E545K 1633 G>A in PIK3CA, 1624G>A in PIK3CA, 3140A>G in PIK3C1, and L858R 2573 T>G in EGFR.

30. The method according to claim 21, wherein the sample is obtained from a human.

31. The method according to claim 22, wherein the denaturing step of the PCR is carried out at a temperature that does not substantially irreversibly inactivate the thermostable restriction endonuclease.

32. The method according to claim 22, wherein the annealing step and/or the extension step of the PCR is carried out at a temperature at which the thermostable restriction endonuclease is active.

33. A method for detecting a mutant allele of a gene, if present, in a sample using a polymerase chain reaction (PCR) comprising:
(a) contacting the sample with a first primer that produces a first and a second amplicon after two rounds of amplification, the first amplicon being an amplicon of the normal allele and cannot serve as a template for subsequent amplification, and the second amplicon being an amplicon of the mutant allele; and
(b) contacting the sample with a second primer that anneals to the second amplicon.

34. The method according to claim 33, wherein the first and the second primers are forward primers.

35. The method according to claim 34, wherein the ratio of the first forward primer to the second forward primer is less than 1:10.

36. The method according to claim 33, wherein the first amplicon forms a hairpin.

37. The method according to claim 33, wherein the first primer is selected from the group consisting of TCTGTCCTGGGATTCTCTTGAGATGTGGTCAATGGAAGAAACCACCAAG (SEQ ID NO: 29), GGGACACTCTAAGATTTTCTTCGCGTTGAAGAAGTACAAAATGTCATTA (SEQ ID NO: 34), and GACAGATTTTCCACTTGCTGTGCGGAAGCTTCATAAGTCAGTCTCATCTGCAAA TAC (SEQ ID NO: 39); and the second primer is selected from the group consisting of CCTGGGATTCTCTTGAGATGTGGTCAAT (SEQ ID NO: 30), ACACTCTAAGATTTTCTTCGCGTT (SEQ ID NO: 35), and ATTTTCCACTTGCTGTGCGGAAGCTTCATA (SEQ ID NO: 40), respectively.

38. The method according claim 33, wherein the mutant allele is selected from the group consisting of 5382 insertion C in BRCA1, 185 deletion AG in BRCA1, and 6174 del T in BRCA2.

39. A template tool for use in selecting optimal PCR conditions for a method of detecting a mutant allele of a gene in a sample, which method selectively degrades a normal allele of the gene in the sample by a thermostable restriction endonuclease, the template tool comprising:
a first polynucleotide sequence comprising recognition sites for a plurality of thermostable endonucleases, and
a second polynucleotide sequence homologous to the first polynucleotide except that it does not contain a single recognition site for any one of the plurality of thermostable endonucleases.

40. The template tool according to claim 39, wherein the first polynucleotide sequence comprises recognition sites for 10-50 different thermostable endonucleases.

41. The template tool according to claim 39, wherein the first polynucleotide sequence comprises recognition sites for 30 different thermostable endonucleases.

42. The template tool according to claim 39, wherein the plurality of thermostable endonucleases are selected from the group consisting of: TasI, Tru1I, TspDTI, BstNI, PspGI, TscAI, TspRI, PhoI, TaaI, BseLI, MwoI, TseI, ApeKI, BtsCI, TfiI, Tsp45I, BstSF1, SmlI, TatI, TaiI, BsiHkAI, BseSI, BstHHI, TaqI, TspGWI, TauI, BclI, BstUI, Tth111I, BseJI, and combinations thereof.

43. The template tool according to claim 39, wherein the recognition site for each of the plurality of thermostable endonucleases is selected from the group consisting of: AATT, TTAA, ATGAA, CCWGG, CCWGG, CASTG, CASTG, GGCC, ACNGT, CCNNNNNNNGG, GCNNNNNNNGC, GCWGC, GCWGC, GGATG, GAWTC, GTSAC, CTRYAG, CTYRAG, WGTACW, ACGT, GWGCWC, GKGCMC, GCGC, TCGA, ACGGA, GCSGC, TGATCA, CGCG, GACNNNGTC, GATNNNNATC, and combinations thereof.

44. The template tool according to claim 39, wherein the first and the second polynucleotide sequences are from about 100 base pairs to about 600 base pairs in length.

45. The template tool according to claim 39, wherein the melting temperature of the first and the second polynucleotide sequences are from about 75° C. to about 90° C.

46. The template tool according to claim 39, wherein the first polynucleotide sequence comprises a sequence as depicted in SEQ ID NO:69 and the second polynucleotide sequence comprises a sequence as depicted in SEQ ID NO:70.

47. A kit comprising the template tool according to claim 39.

48. A kit comprising the template tool according to claim 46.

49. The method according to claim 22, wherein conditions for the PCR are selected by using a template tool comprising:
a first polynucleotide sequence comprising recognition sites for a plurality of thermostable endonucleases, and
a second polynucleotide sequence homologous to the first polynucleotide except that it does not contain a single recognition site for any one of the plurality of thermostable endonucleases.

50. A template tool for use in selecting optimal PCR conditions for a method of selectively amplifying mutant alleles present in low abundance in solid tumors, which method selectively degrades a normal allele or an amplicon of the normal allele in the sample by a thermostable restriction endonuclease, the template tool comprising:
a first polynucleotide sequence comprising recognition sites for a plurality of thermostable endonucleases, and
a second polynucleotide sequence homologous to the first polynucleotide except that it does not contain a single recognition site for any one of the plurality of thermostable endonucleases.

51. The template tool according to claim 50, wherein the first polynucleotide sequence comprises recognition sites for 30 different thermostable endonucleases.

52. The template tool according to claim 50, wherein the plurality of thermostable endonucleases are selected from the group consisting of: TasI, Tru1I, TspDTI, BstNI, PspGI, TscAI, TspRI, PhoI, TaaI, BseLI, MwoI, TseI, ApeKI, BtsCI, TfiI, Tsp45I, BstSF1, SmlI, TatI, TaiI, BsiHkAI, BseSI, BstHHI, TaqI, TspGWI, TauI, BclI, BstUI, Tth111I, BseJI, and combinations thereof.

53. The template tool according to claim 50, wherein the recognition site for each of the plurality of thermostable endonucleases is selected from the group consisting of: AATT, TTAA, ATGAA, CCWGG, CCWGG, CASTG, CASTG, GGCC, ACNGT, CCNNNNNNNGG, GCNNNNNNNGC, GCWGC, GCWGC, GGATG, GAWTC, GTSAC, CTRYAG, CTYRAG, WGTACW, ACGT, GWGCWC, GKGCMC, GCGC, TCGA, ACGGA, GCSGC, TGATCA, CGCG, GACNNNGTC, GATNNNNATC, and combinations thereof.

54. The template tool according to claim 50, wherein the first polynucleotide sequence comprises a sequence as depicted in SEQ ID NO:69 and the second polynucleotide sequence comprises a sequence as depicted in SEQ ID NO:70.

55. A kit comprising the template tool according to claim 50.

56. A kit comprising the template tool according to claim 54.


SEQUENCE LISTING (70 sequences)

SEQ ID NO: 1 - DNA, 46 bp, Artificial
  Synthetic Construct
cgttgaagaagtacaaaatgtcattaatgctatgcagaaaagctta

SEQ ID NO: 2 - DNA, 28 bp, Artificial
  Synthetic Construct
cttgtgctgacttaccagatggggcact

SEQ ID NO: 3 - DNA, 11 bp, Artificial
  Synthetic Construct
gcttagagtgc

SEQ ID NO: 4 - DNA, 9 bp, Artificial
  Synthetic Construct
gcttagtgc

SEQ ID NO: 5 - DNA, 40 bp, Artificial
  Synthetic Construct
aatggaagaaaccaccaaggtccaaagcgagcaagagcat

SEQ ID NO: 6 - DNA, 33 bp, Artificial
  Synthetic Construct
acttgagggagggagctttacctttctggcctg

SEQ ID NO: 7 - DNA, 11 bp, Artificial
  Synthetic Construct
gcatcccaggc

SEQ ID NO: 8 - DNA, 12 bp, Artificial
  Synthetic Construct
gcatccccaggc

SEQ ID NO: 9 - DNA, 34 bp, Artificial
  Synthetic Construct
ctgcaaatacttgtgggatttttagcacagccag

SEQ ID NO: 10 - DNA, 35 bp, Artificial
  Synthetic Construct
gacttgcttggtactatcttctatttcagaaaaca

SEQ ID NO: 11 - DNA, 5 bp, Artificial
  Synthetic Construct
cagtg

SEQ ID NO: 12 - DNA, 5 bp, Artificial
  Synthetic Construct
caggg

SEQ ID NO: 13 - DNA, 41 bp, Artificial
  Synthetic Construct
gctgaaaatgactgaatataaacttgtggtagttggaactg

SEQ ID NO: 14 - DNA, 35 bp, Artificial
  Synthetic Construct
atattcgtccacaaaatgattctgaattagctgta

SEQ ID NO: 15 - DNA, 6 bp, Artificial
  Synthetic Construct
actggg

SEQ ID NO: 16 - DNA, 6 bp, Artificial
  Synthetic Construct
actgag

SEQ ID NO: 17 - DNA, 35 bp, Artificial
  Synthetic Construct
ttcttcatgaagacctcacagtaaaaataggtgat

SEQ ID NO: 18 - DNA, 33 bp, Artificial
  Synthetic Construct
acaaaatggatccagacaactgttcaaactgat

SEQ ID NO: 19 - DNA, 5 bp, Artificial
  Synthetic Construct
cagtg

SEQ ID NO: 20 - DNA, 5 bp, Artificial
  Synthetic Construct
cagag

SEQ ID NO: 21 - DNA, 28 bp, Artificial
  Synthetic Construct
gagacaatgaattaagggaaaatgacaa

SEQ ID NO: 22 - DNA, 5 bp, Artificial
  Synthetic Construct
cactg

SEQ ID NO: 23 - DNA, 26 bp, Artificial
  Synthetic Construct
ctgtgactccatagaaaatctttctc

SEQ ID NO: 24 - DNA, 5 bp, Artificial
  Synthetic Construct
cacta

SEQ ID NO: 25 - DNA, 47 bp, Artificial
  Synthetic Construct
cgtactggtgaaaacaccgcagcatgtcaagatcacagattttggcc

SEQ ID NO: 26 - DNA, 5 bp, Artificial
  Synthetic Construct
cctgg

SEQ ID NO: 27 - DNA, 34 bp, Artificial
  Synthetic Construct
gtgtaaaattgattccaatgccatccacttgata

SEQ ID NO: 28 - DNA, 5 bp, Artificial
  Synthetic Construct
ccggg

SEQ ID NO: 29 - DNA, 49 bp, Artificial
  Synthetic Construct
tctgtcctgggattctcttgagatgtggtcaatggaagaaaccaccaag

SEQ ID NO: 30 - DNA, 28 bp, Artificial
  Synthetic Construct
cctgggattctcttgagatgtggtcaat

SEQ ID NO: 31 - DNA, 25 bp, Artificial
  Synthetic Construct
cccatctctgcaaaggggagtggaa

SEQ ID NO: 32 - DNA, 81 bp, Artificial
  Synthetic Construct
tctgtcctgggattctcttgagatgtggtcaatggaagaaaccaccaaggtccaaagcgagcaagagaatcccaggacaga

SEQ ID NO: 33 - DNA, 82 bp, Artificial
  Synthetic Construct
tctgtcctgggattctcttgagatgtggtcaatggaagaaaccaccaaggtccaaagcgagcaagagaatccccaggacaga

SEQ ID NO: 34 - DNA, 49 bp, Artificial
  Synthetic Construct
gggacactctaagattttcttcgcgttgaagaagtacaaaatgtcatta

SEQ ID NO: 35 - DNA, 24 bp, Artificial
  Synthetic Construct
acactctaagattttcttcgcgtt

SEQ ID NO: 36 - DNA, 31 bp, Artificial
  Synthetic Construct
aggtcaattctgttcatttgcataggagata

SEQ ID NO: 37 - DNA, 78 bp, Artificial
  Synthetic Construct
gggacactctaagattttcttcgcgttgaagaagtacaaaatgtcattaatgctatgcagaaaatcttagagtgtccc

SEQ ID NO: 38 - DNA, 76 bp, Artificial
  Synthetic Construct
gggacactctaagattttcttcgcgttgaagaagtacaaaatgtcattaatgctatgcagaaaatcttagtgtccc

SEQ ID NO: 39 - DNA, 57 bp, Artificial
  Synthetic Construct
gacagattttccacttgctgtgcggaagcttcataagtcagtctcatctgcaaatac

SEQ ID NO: 40 - DNA, 30 bp, Artificial
  Synthetic Construct
attttccacttgctgtgcggaagcttcata

SEQ ID NO: 41 - DNA, 37 bp, Artificial
  Synthetic Construct
gtactatcttctatttcagaaaacacttgtcttgcgt

SEQ ID NO: 42 - DNA, 93 bp, Artificial
  Synthetic Construct
gacagattttccacttgctgtgcggaagcttcataagtcagtctcatctgcaaatactgtgggatttttagcacagcaagtggaaaatctgtc

SEQ ID NO: 43 - DNA, 92 bp, Artificial
  Synthetic Construct
gacagattttccacttgctgtgcggaagcttcataagtcagtctcatctgcaaatactgtgggatttttagcacagcaagggaaaatctgtc

SEQ ID NO: 44 - DNA, 42 bp, Artificial Sequence
  Synthetic construct
tctctttgatgtcacgttcaccaccacagctgagagggaaat

SEQ ID NO: 45 - DNA, 22 bp, Artificial Sequence
  Synthetic construct
tctctttgatgtcacgttcacc

SEQ ID NO: 46 - DNA, 24 bp, Artificial Sequence
  Synthetic construct
ggaattgaatgtagtttcatggat

SEQ ID NO: 47 - DNA, 1889 bp, Mus musculus
ctgtcgagtcgcgtccacccgcgagcacagcttctttgcagctccttcgttgccggtccacacccgccaccagttcgccatggatgacgatatcgctgcgctggtcgtcgacaacggctccggcatgtgcaaagccggcttcgcgggcgacgatgctccccgggctgtattcccctccatcgtgggccgccctaggcaccagggtgtgatggtgggaatgggtcagaaggactcctatgtgggtgacgaggcccagagcaagagaggtatcctgaccctgaagtaccccattgaacatggcattgttaccaactgggacgacatggagaagatctggcaccacaccttctacaatgagctgcgtgtggcccctgaggagcaccctgtgctgctcaccgaggcccccctgaaccctaaggccaaccgtgaaaagatgacccagatcatgtttgagaccttcaacaccccagccatgtacgtagccatccaggctgtgctgtccctgtatgcctctggtcgtaccacaggcattgtgatggactccggagacggggtcacccacactgtgcccatctacgagggctatgctctccctcacgccatcctgcgtctggacctggctggccgggacctgacagactacctcatgaagatcctgaccgagcgtggctacagcttcaccaccacagctgagagggaaatcgtgcgtgacatcaaagagaagctgtgctatgttgctctagacttcgagcaggagatggccactgccgcatcctcttcctccctggagaagagctatgagctgcctgacggccaggtcatcactattggcaacgagcggttccgatgccctgaggctcttttccagccttccttcttgggtatggaatcctgtggcatccatgaaactacattcaattccatcatgaagtgtgacgttgacatccgtaaagacctctatgccaacacagtgctgtctggtggtaccaccatgtacccaggcattgctgacaggatgcagaaggagattactgctctggctcctagcaccatgaagatcaagatcattgctcctcctgagcgcaagtactctgtgtggatcggtggctccatcctggcctcactgtccaccttccagcagatgtggatcagcaagcaggagtacgatgagtccggcccctccatcgtgcaccgcaagtgcttctaggcggactgttactgagctgcgttttacaccctttctttgacaaaacctaacttgcgcagaaaaaaaaaaaataagagacaacattggcatggctttgtttttttaaattttttttaaagtttttttttttttttttttttttttttttaagtttttttgttttgttttggcgcttttgactcaggatttaaaaactggaacggtgaaggcgacagcagttggttggagcaaacatcccccaaagttctacaaatgtggctgaggactttgtacattgttttgtttttttttttttttggttttgtctttttttaatagtcattccaagtatccatgaaataagtggttacaggaagtccctcaccctcccaaaagccacccccactcctaagaggaggatggtcgcgtccatgccctgagtccaccccggggaaggtgacagcattgcttctgtgtaaattatgtactgcaaaaatttttttaaatcttccgccttaatacttcatttttgtttttaatttctgaatggcccaggtctgaggcctcccttttttttgtccccccaacttgatgtatgaaggctttggtctccctgggagggggttgaggtgttgaggcagccagggctggcctgtacactgacttgagaccaataaaagtgcacaccttaccttacacaaac

SEQ ID NO: 48 - DNA, 1296 bp, Rattus norvegicus
ggggtcgagtccgcgtccacccgcgagtacaaccttcttgcagctcctccgtcgccggtccacacccgccaccagttcgccatggatgacgatatcgctgcgctcgtcgtcgacaacggctccggcatgtgcaaggccggcttcgcgggcgacgatgctccccgggccgtcttcccctccatcgtgggccgccctaggcaccagggtgtgatggtgggtatgggtcagaaggactcctacgtgggcgacgaggcccagagcaagagaggcatcctgaccctgaagtaccccattgaacacggcattgtcaccaactgggacgatatggagaagatttggcaccacactttctacaatgagctgcgtgtggcccctgaggagcaccctgtgctgctcaccgaggcccctctgaaccctaaggccaaccgtgaaaagatgacccagatcatgtttgagaccttcaacaccccagccatgtacgtagccatccaggctgtgttgtccctgtatgcctctggtcgtaccactggcattgtgatggactccggagacggggtcacccacactgtgcccatctatgagggttacgcgctccctcatgccatcctgcgtctggacctggctggccgggacctgacagactacctcatgaagatcctgaccgagcgtggctacagcttcaccaccacagctgagagggaaatcgtgcgtgacattaaagagaagctgtgctatgttgccctagacttcgagcaagagatggccactgccgcatcctcttcctccctggagaagagctatgagctgcctgacggtcaggtcatcactatcggcaatgagcggttccgatgccccgaggctctcttccagccttccttcctgggtatggaatcctgtggcatccatgaaactacattcaattccatcatgaagtgtgacgttgacatccgtaaagacctctatgccaacacagtgctgtctggtggcaccaccatgtacccaggcattgctgacaggatgcagaaggagattactgccctggctcctagcaccatgaagatcaagatcattgctcctcctgagcgcaagtactctgtgtggattggtggctctatcctggcctcactgtccaccttccagcagatgtggatcagcaagcaggagtacgatgagtccggcccctccatcgtgcaccgcaaatgcttctaggcggactgttactgagctgcgttttacaccctttctttgacaaaacctaacttgcgcaaaaaaaaaaaaaaaaaaaaaaaaaaaaaa

SEQ ID NO: 49 - DNA, 30 bp, Artificial Sequence
  Synthetic construct
catttacatctttcaattatgtatattctt

SEQ ID NO: 50 - DNA, 30 bp, Artificial Sequence
  Synthetic construct
atgcaaatacttcattagtaaaatttactt

SEQ ID NO: 51 - DNA, 35 bp, Artificial Sequence
  Synthetic Construct
cttattgactctaagaggaaagatgaagtactatg

SEQ ID NO: 52 - DNA, 30 bp, Artificial Sequence
  Synthetic Construct
caattcttaccatccacaaaatggatccag

SEQ ID NO: 53 - DNA, 35 bp, Artificial Sequence; identical to SEQ ID NO: 51
  Synthetic Construct

SEQ ID NO: 54 - DNA, 30 bp, Artificial Sequence; identical to SEQ ID NO: 52
  Synthetic Construct

SEQ ID NO: 55 - DNA, 34 bp, Artificial Sequence; identical to SEQ ID NO: 9
  Synthetic Construct

SEQ ID NO: 56 - DNA, 35 bp, Artificial Sequence; identical to SEQ ID NO: 10
  Synthetic Construct

SEQ ID NO: 57 - DNA, 28 bp, Artificial Sequence
  Synthetic Construct
gagacaatgaattaagggaaaatgacaa

SEQ ID NO: 58 - DNA, 26 bp, Artificial Sequence
  Synthetic Construct
ctgtgactccatagaaaatctttctc

SEQ ID NO: 59 - DNA, 41 bp, Artificial Sequence; identical to SEQ ID NO: 13
  Synthetic Construct

SEQ ID NO: 60 - DNA, 35 bp, Artificial Sequence; identical to SEQ ID NO: 14
  Synthetic Construct

SEQ ID NO: 61 - DNA, 35 bp, Artificial Sequence
  Synthetic Construct
gactgaatataaacttgtggtagttggcgctggtg

SEQ ID NO: 62 - DNA, 35 bp, Artificial Sequence
  Synthetic Construct
agagaaacctttatctgtatcaaagaatggtcctg

SEQ ID NO: 63 - DNA, 30 bp, Artificial Sequence
  Synthetic Construct
agaccctagccttagataaaactgagcaag

SEQ ID NO: 64 - DNA, 30 bp, Artificial Sequence
  Synthetic Construct
tcaatgcatgctgtttaattgtgtggaaga

SEQ ID NO: 65 - DNA, 26 bp, Artificial Sequence
  Synthetic Construct
ttgactctatgatgaaagatgttgtt

SEQ ID NO: 66 - DNA, 25 bp, Artificial Sequence
  Synthetic Construct
attacgatccacaaaatgtatgcag

SEQ ID NO: 67 - DNA, 390 bp, Artificial Sequence
  Synthetic Construct
agacatacttattgactctaagaggaaagatgaagtactatgttttaaagaatattatattacagaattatagaaattagatctcttacctaaactcttcataaagcttgctctgataggaaaatgagatctactgttttcctttacttactacacctcagatatatttcttcatgaagacctcacagtaaaaataggtgattttggtctagctacagtgaaatctcgatggagtgagtcccatcagtttgaacagttgtctggatccattttgtggatggtaagaattgaggctatttttccactgattaaatttttggccctgagatgctgctgagttactagaaagtcattgaaggtctcaactatagtattttcatagttcccagt

SEQ ID NO: 68 - DNA, 390 bp, Artificial Sequence
  Synthetic Construct
agacatacttattgactctaagaggaaagatgaagtactatgttttaaagaatattatattacagaattatagaaattagatctcttacctaaactcttcataatgcttgctctgataggaaaatgagctctactgttttcctttacttactacacctcagatatatttcttcatgaagacctcacagtaaaaataggtgattttggtctagctacagagaaatctcgatggagtgggtcccatcagtttgaacagctgtctggatccattttgtggatggtaagaattgaggctatttttccactgattaaatttttggccctgagatgctgctgagttactagaaagtcattgaaggtctcaactatagtattttcatagttcccagt

SEQ ID NO: 69 - DNA, 306 bp, Artificial Sequence
  Synthetic Construct
ttgactctatgatgaaagatgttgttccaggatataattcactgatatggccatatttaaacagtccaaataatgggctgcattgctttattagcggatgtatagattcatagtgacctatagtaaatgcattgtacttatgtgcacatgaaatagcgctattcgaacaaatactccatactcaagatataatgaggagctgaaccacgacgagacataacggaatatgccgcattaatgatcattacgcgttagacaatgtcaagattataatcatacgtctgcatacattttgtggatcgtaat

SEQ ID NO: 70 - DNA, 306 bp, Artificial Sequence
  Synthetic Construct
ttgactctatgatgaaagatgttgttccacgatatatttctctgatatgcccatattataagagtgcaaataatgggctccattcctttattagcggttgtatagtttcatagagaccaatagtaagatatctgaacttatgtccacatgtaatagggctatacgaacaaatagtccatacacaagatataatgacgaggagaagcaccaccagacatatcggaatatgccggattaatgttcattacgccttacacaatgtcaagtttataatcataggtctgcatacattttgtggatcgtaat